(19) United States
(12) Patent Application Publication
(10) Pub. No.: US 2021/0024893 A1
(43) Pub. Date: Jan. 28, 2021

(54) MEDICAL USES OF EXOSOMES

(71) Applicants: Kayvan NIAZI, (US); VBC HOLDINGS LLC, Culver City, CA (US)

(72) Inventors: Kayvan NIAZI, Encino, CA (US); Francesco CURCIO, Pagnacco (UD) (IT)

(73) Assignee: VBC HOLDINGS LLC, Culver City, CA (US)

(21) Appl. No.: 16/341,793

(22) PCT Filed: Oct. 12, 2017
(86) PCT No.: PCT/US2017/056350

§ 371 Date: Apr. 12, 2019

Related U.S. Application Data
(60) Provisional application No. 62/407,831, filed on Oct. 13, 2016.
(60) Provisional application No. 62/544,979, filed on Aug. 14, 2017.

(51) Int. Cl. C12N 5/0775; A61K 31/7088

(52) U.S. Cl. CPC C12N 5/0667; A61K 31/7088; C12N 5/0663; A61K 9/0073; C12N 2501/24; C12N 2501/25; C12N 5/0668

(57) ABSTRACT

The invention provides anti-inflammatory exosomes that, when administered locally or systemically, downregulate an inflammatory process in a subject in need of such treatment, as well as methods of making and using the anti-inflammatory exosomes.

### TECHNICAL FIELD

The present disclosure relates generally to anti-inflammatory exosomes, methods of obtaining or producing anti-inflammatory exosomes, and to methods of treating a disease or disorder exhibiting or caused by an inflammatory process, by administering anti-inflammatory exosomes to a subject needing such treatment.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Inflammation is a normal response of the immune system to a wide variety of injuries, infection and/or other insults to living tissue. Generally, inflammation results from an acute injury or disease process, and the signs of inflammation, e.g., pain, heat, redness, swelling, and loss of function, are of limited scope and duration. The inflammatory reaction is mediated by a complex interplay of a variety of immune cells and chemical mediators, such as bradykinin and histamine, as well as various cytokines. Unfortunately, some types of injury and/or disease processes, particularly those that are long lasting and chronic in nature, can provoke a corresponding long lasting inflammatory process in living tissue that will cause further damage to the affected and surrounding tissues, organs, or the entire organism.

Chronic inflammation is associated with, or found to cause a wide range of disease processes, both localized and systemic, in nearly every organ system. For example, metabolic syndrome (also referred to as, “metabolic X syndrome”) is a chronic condition that can occur in mammals, including humans, that exhibit chronic above normal central fat deposits and that receive insufficient exercise. Metabolic syndrome is a systemic inflammatory condition associated with elevated levels of acute-phase proteins, e.g., C-reactive protein (“CRP”). Metabolic syndrome is also associated with an increased risk of coronary artery disease, e.g., atherosclerosis and ischemic heart disease, type 2 diabetes, diseases of other end artery organs, peripheral artery disease and related conditions.

Diseases of the respiratory system are also caused by, or exacerbated by, chronic inflammation. These include, for example, asthma, bronchitis and chronic obstructive pulmonary disease (COPD). CRP is also a marker associated with systemic inflammation in COPD.

In addition, chronic inflammation is implicated in a number of diseases of the gut, such as inflammatory bowel disease or IBD. IBD includes ulcerative colitis and Crohn's disease. Skin diseases associated with inflammation include, for example, dermatitis, eczema and psoriasis. It has also been suggested that a number of diseases of the central nervous system, including Alzheimer's disease, and Parkinson's disease, are caused by, or exacerbated by, chronic inflammatory processes. Certain diseases of the musculature are also caused by, or exacerbated by, chronic inflammation, e.g., polymyositis, dermatomyositis (affects skin and muscle), inclusion body myositis (IBM) and juvenile myositis. Diseases of the joints that are caused by, or exacerbated by, chronic inflammation, such as rheumatoid arthritis, osteoarthritis, amyloidosis, ankylosing spondylitis, bursitis, psoriatic arthritis, Still's disease and others.

Osteoarthritis is the most common form of arthritis in humans, and is thought to initially result from the breakdown of joint cartilage. However, as the disease progresses, the degenerative effects of osteoarthritis disease process extend into the bone as well.

To date, medical treatments for chronic inflammatory conditions mainly include administering anti-inflammatory medications, such as steroidal or nonsteroidal anti-inflammatory agents. These are administered in order to reduce pain and inflammation. Sometimes analgesics, e.g., acetaminophen and/or opiates are also administered to enhance pain relief. Steroidal anti-inflammatory medications, such as betamethasone, methylprednisolone, triamcinolone, and the hundreds of analogous medications, can be administered topically, orally, by systemic injection, such as intramuscularly, intravenously, and/or by direct injection or infusion into the impacted tissue, or by inhalation for pulmonary conditions.

Non-steroidal anti-inflammatory agents (NSAIDs) can be can be administered systemically, such as orally, intramuscularly and/or intravenously, as well as topically. NSAIDs include, for example, aspirin, and its derivatives, ibuprofen, ketorolac, flurbiprofen, celecoxib, etodolac and naproxen, to name but a few such medications. Both anti-inflammatory medications and analgesics are sometimes of limited long term effectiveness, and both short and long term use of these medications raises the risk of potentially serious side effects.

Chronic inflammation has also been associated with creating a predisposition or increased risk of developing certain types of precancerous conditions (e.g., hyperplasia, metaplasia, dysplasia), and/or cancers. For example, see the review article by Schacter, et al., 2002, Oncology 16:217-26, and particularly the list of inflammatory conditions predisposing to cancer provided by Schacter et al. at Table 1. For example, gastritis caused by H. pylori is associated with a risk of gastric adenocarcinoma, chronic cholecystitis caused by certain bacteria and/or stone formation is associated with a risk of gall bladder cancer, inflammatory bowel disease is associated with a risk of colorectal carcinoma, and asbestosis or silicosis is associated with a risk of mesothelioma or lung cancer.

In one attempt at providing improved methods of treating inflammation, clinical trials by Jo et al., 2014, Stem Cells 32(5):1254-66. doi: 10.1002/stem.1634; Wyles et al. 2015, Stem Cells Cloning.28;8:117-24. doi: 10.2147/SCCAA.S68073.) have demonstrated that local injection of adipose mesenchymal stem cells (AMSC) into osteoarthritic joints provided improved function and are likely to inhibit osteophyte formation and reduce cartilage degeneration. The anti-inflammatory properties of AMSC have been linked to their immunosuppressive potential. It has been proposed that one type of mediator of the immunosuppressive potential of AMSC are exosomes (Maumus et al., 2013 Biochimie. 95(12):2229-34. doi: 10.1016/j.biochi.2013.04.017. Vishnubhatla et al., 2014 J Circ Biomark; 3:2 doi 10.5772/58597).

Exosomes are small membrane-bound particles secreted by most cell types, including stem cells, in organisms across a wide taxonomic range (Yu et al., 2014, Int J Mol Sci. 7;15(3):4142-57. doi: 10.3390/ijms15034142.). Exosomes originate from internal budding of the cellular plasma membrane during endocytotic internalization, from cellular structures identified as multivesicular endosomes (MVE), that package cytoplasmic materials as membrane-bound vesicles. Exosomes have been variously reported to range in diameter from as broadly as from 30 to about 200 nm, to more particularly from about 40 to about 100 nm. Exosomes have been found to facilitate the delivery and the transfer of proteins, lipids and nucleic acids between cells. Exosomes are released from both normal and diseased cells, and are found in blood and other bodily fluids.

Exosomes have previously been shown to mediate both immunostimulatory (Zitvogel et al., US20040028692) and immunoinhibitory modulation of the immune system. Whiteside et al. 2005, British Journal of Cancer 92: 209-211). Robbins et al., US20060116321, describe the immune inhibiting properties of exosomes derived from dendritic cells.

However, there remains a longstanding need in the art for improved agents for treating chronic inflammatory conditions, as well a need for improved methods of obtaining and using the same.

### SUMMARY OF THE INVENTION

Accordingly, the invention provides for methods of making anti-inflammatory exosomes, methods of treating inflammation by administering the anti-inflammatory exosomes, and for isolated and purified exosomes produced by the inventive methods

In a first embodiment, the invention provides a method of producing anti-inflammatory exosomes capable of inhibiting inflammation in a subject diagnosed with an inflammatory disease or disorder, the exosomes produced by a process comprising:

(a) culturing adult stem cells in a suitable culture medium, for a time period sufficient for the adult stem cell culture medium to accumulate a useful concentration of anti-inflammatory exosomes, wherein the culture medium comprises an activating composition,

(b) collecting the culture medium of (a), and

(c) isolating anti-inflammatory adult stem cell exosomes from the culture medium collected in (b);

wherein the activating composition comprises at least one of:

(i) fluid extracted from inflamed tissue or other anatomical structure of a mammal diagnosed with the inflammatory disease or disorder,

(ii) blood, plasma or serum from a mammal diagnosed with the inflammatory disease or disorder,

(iii) the fluid of (i) or (ii) further comprising at least one cytokine at a concentration sufficient to enhance the effectiveness of the activating composition comprising (i) or (ii),

(iv) one or more cytokines at a concentration sufficient to induce the adult stem cells to secrete anti-inflammatory exosomes capable of inhibiting inflammation in a mammal, and the activating composition excludes the fluid of (i) and/or the blood, plasma or serum of (ii).

Preferably, the at least one cytokine of (iii) or (iv) is selected from the group consisting of interferon-gamma (IFNγ), interleukin-1α (IL-1α), interleukin-1-β (1IL-1β), interleukin-6 (IL-6), interleukin 12b (IL-12b), tumor necrosis factor-α (TNFα) and combinations thereof, and is present in a concentration ranging from about 10 ng/ml to about 50 ng/ml or from about 10 ng/ml to about 40 ng/ml. Optionally, the cytokine of (iii) and/or (iv) is from an exogenous source.

In one particular embodiment, the cytokine is IFNγ and/or TNFα.

In another embodiment, the time period for the culturing of step (a) ranges from about 3 to 6 days, from about 24 to about 72 hours or from about 24 to about 48 hours.

Further, the anti-inflammatory adult stem cell exosomes are isolated from the culture medium of (a) by polymer precipitation, immunological separation, magnetic immunocapture, ultracentrifugation, density gradient centrifugation, size exclusion chromatography, and/or ultrafiltration.

In a second embodiment, the invention provides a method of treating a subject diagnosed with an inflammatory disease or disorder, comprising administering to the subject an effective amount of the anti-inflammatory exosomes of claim 1.

Preferably, the invention provides a method of treating the inflammatory diseases resulting from metabolic X syndrome, inflammatory diseases of the gastrointestinal system, inflammatory diseases of the pulmonary system, inflammatory diseases of the skin, inflammatory diseases of the musculature, inflammatory diseases of the joints, and/or inflammatory diseases of the nervous system. In particular, the invention provides a method of treating tissue specific disorders, including, inflammation associated with coronary artery disease, chronic obstructive pulmonary disease (COPD), asthma, bronchitis, inflammatory bowel disease (IBD), Alzheimer's disease, Parkinson's disease, polymyositis, dermatomyositis, inclusion body myositis (IBM), juvenile myositis, rheumatoid arthritis, osteoarthritis, amyloidosis, ankylosing spondylitis, bursitis, psoriatic arthritis, Still's disease, and/or precancerous conditions.

When the inflammatory disease or disorder is osteoarthritis, the method of treating comprises either systemic injection of the anti-inflammatory exosomes and/or injection of an effective amount of the anti-inflammatory exosomes into one or more inflamed joints of the subject.

When the inflammatory disease or disorder is pulmonary in nature, the method comprises either systemic injection of the anti-inflammatory exosomes and/or administering the anti-inflammatory exosomes as an inhaled mist or aerosol.

Preferably, the subject to be treated is a mammal such as a human, a canine, equine, feline and/or porcine subject in need thereof. These include domestic dogs, horses, cats, pigs and the like.

The invention also includes methods for administering the anti-inflammatory exosomes. The anti-inflammatory exosomes are administered systemically, in an amount effective to inhibit or suppress the inflammatory response in a subject. An effective amount ranges, for example, from about 1.5×10¹⁰ to about 1.5×10¹³ exosome particles per kilogram of total body weight. Alternatively, the effective amount of anti-inflammatory exosomes that is administered ranges, for example, from about 1.5×10¹⁰ and 1.5×10¹¹ exosome particles injected or infused into a localized tissue or anatomical space.

In an optional alternative embodiment, it is contemplated to co-treat the subject with at least one additional type of anti-inflammatory agent, wherein the at least one additional anti-inflammatory agent is selected from the group consisting of a steroidal anti-inflammatory, a non-steroidal anti-inflammatory, an anti-inflammatory anti-TNF alpha antibody and/or combinations thereof.

In a third embodiment, the invention provides isolated and purified exosomes produced by the inventive method, as well as pharmaceutical compositions that include the inventive exosomes, plus physiologically compatible solvents, carriers and/or excipients for optimal storage and administration of the isolated and purified exosomes. Optionally, the isolated and purified anti-inflammatory exosomes include, for example, least one miRNA including miRNA-34, miRNA-146 and/or miR-127. In an alternative option, the isolated and purified anti-inflammatory exosomes have an miRNA content that consists essentially of at least one miRNA selected from the group consisting of miRNA-34, miRNA-146 and miR-127

### BRIEF DESCRIPTION OF THE DRAWINGS

FIG. 1A illustrates the expression of exosomal markers: CD9, CD63, CD81, and the protein expressed by tumor susceptibility gene 101 (“TGS101”) in synovial fluid derived exosomes, by Western blot.

FIG. 1B illustrates a latex bead for the flow cytometry determination.

FIG. 1C illustrates a scatter chart of SSC-A (Y-axis, side-scattered light) verses FSC-A (X-axis, forward-scattered light) of the latex beads conjugated with anti-CD63 antibody. The R1 gate (a software filter) is plotted on single beads, excluding beads aggregates. The graph represents the cytofluorimetric analysis of the beads which are analyzed based on their light scatter properties. Forward-scattered light (FSC) is proportional to particles-surface area or size, while side-scattered light (SSC) is proportional to particles granularity or internal complexity.

FIG. 1D illustrates the expression of exosomal markers: CD7, CD9, and DC81 in synovial fluid derived exosomes, by flow cytometry, based on the bead's R¹ gate.

FIG. 1E illustrates the quantification of exosomes by determination of acetyl-CoA esterase (AChE) activity with an Exocet™ kit, showing the optical density (Y-axis) of the AChE assay verses the number of exosomes. The figure reports the linear regression equation and the R² (coefficient of determination) which is the proportion of the variance in the dependent variable that is predictable from the independent variable.

FIG. 1F normalizes the number of exosomes as 10⁷ exosomes per microliter.

FIG. 2A illustrates the immunoblots obtained for a comparison between patient respective serum derived exosomes and respective synovial fluid derived exosomes by Western blot analysis for markers CD9, CD63, CD81, and TGS101.

FIG. 2B illustrates arbitrary units of CD9 from serum (S), verses units of CD9 from synovial fluid (SF). Arbitrary Units express the band intensity of Western blots and are measured by densitometry analysis with ImageJ software (open source Java based image processing software developed at the U.S. National Institutes of Health and available without cost by downloading from various sites on the internet). They are the same units for both type of exosomes.

FIG. 2C illustrates arbitrary units of CD63 from serum (S), verses units of CD63 from synovial fluid (SF).

FIG. 2D illustrates arbitrary units of TGS101 from serum (S), verses units of TGS101 from synovial fluid (SF).

FIG. 2E illustrates arbitrary units of CD81 from serum (S), verses units of CD81 from synovial fluid (SF).

FIG. 3A illustrates a bar graph comparison between CD9 expression in respective patient serum derived exosomes and respective synovial fluid derived exosomes. “MFI” is Mean Fluorescence Intensity.

FIG. 3B illustrates a bar graph comparison between CD81 expression in respective patient serum derived exosomes and respective synovial fluid derived exosomes.

FIG. 4A illustrates the production of M1 macrophages from M0 monocytes in the presence of GM-CSF.

FIG. 4B illustrates percent of cytokine production with respect to untreated control with serum (S) versus synovial fluid (SF) derived exosomes. The cytokines were produced by M1 macrophages treated with SF-derived exosomes

FIG. 5A illustrates the effect of synovial fluid-derived exosomes on interleukin 1 beta (IL-10) mRNA expression in M1 macrophages, by reverse transcription-polymerase chain reaction (“RT-PCR”).

FIG. 5B illustrates the effect of synovial fluid-derived exosomes on interleukin 6 (IL-6) mRNA expression in M1 macrophages, by RT-PCR.

FIG. 5C illustrates the effect of synovial fluid-derived exosomes on tumor necrosis factor alpha (“TNFα”) mRNA expression in M1 macrophages, by RT-PCR.

FIG. 5D illustrates the effect of synovial fluid-derived exosomes on interleukin 12b (IL-12b) mRNA expression in M1 macrophages, by RT-PCR.

FIG. 6A represents an electrophoresis gel with Coomassie blue staining of synovial fluid derived exosome samples prepared by polymer precipitation (Exoquick™) subjected to electrophoresis, confirming IgG antibody light and heavy chain co-precipitation with exosomes.

FIG. 6B illustrates a gel prepared by immunofixation, also confirming immune complex contamination of exosomes by IgG antibody light and heavy chains. Immunofixation electrophoresis (IFE) identificates immunoglobulins, which are separated by electrophoresis followed by precipitation with specific antibodies in situ.

FIG. 7A illustrates a gel evaluating potential immune complex by immunofixation, comparing exosomes isolated by polymer precipitation (left) and exosomes isolated by immunocapture (right). Exosomes were first isolated by polymer precipitation and then were purified by immunocapture.

FIG. 7B illustrates a gel evaluating expression of marker TGS101 by Western blot.

FIG. 7C illustrates Exo-FITC™ (fluorescein) staining by flow cytometry.

FIG. 7D illustrates cytofluorimetic histograms of the exosome-bound beads stained with Exo-FITCTm. The histograms of the beads stained with Exo-FITC™ were also reported as control for the analysis.

FIG. 8 illustrates the effect of purified exosomes on expression of mRNA that expresses cytokines in M1 macrophages, by RT-PCR

FIG. 9A illustrates the effect of synovial fluid from the joints of osteoarthritic patient on the proliferation of adipose derived mesenchymal stem cells (“AMSC”) (n-5) (means±S.D.) The synovial fluid was diluted 1:2 and 1:5 with AMSC culture medium.

FIG. 9B illustrates the effect of synovial fluid from the joints of osteoarthritic patient on the viability of AMSC (n-5) (means±S.D.) The synovial fluid was diluted 1:2 and 1:5 with AMSC culture medium.

FIG. 10 summarizes testing results for the immunophenotype of AMSC that were exposed to synovial fluid from inflamed joints of osteoarthritis patients. Testing was by flow cytometry.

FIG. 11A illustrates results for the CXCLS (a/k/a C-X-C motif chemokine 5 or ENA-78) production by AMSC that were exposed to synovial fluid from inflamed joints of osteoarthritis patients.

FIG. 11B illustrates results for CX3CL1 (a/k/a C-X3-CL motif 1 or Fracktalkine) production by AMSC that were exposed to synovial fluid from inflamed joints of osteoarthritis patients.

FIG. 11C illustrates results for CXCL6 (a/k/a C-X-C motif ligand 6 or granulocyte chemotactic protein 2 [GCP-2[) production by AMSC that were exposed to synovial fluid from inflamed joints of osteoarthritis patients.

IG. 11D illustrates results for CXCL1 (a/k/a C-X-C motif ligand 1, GRO1, GROa or GRO-Alpha) production by AMSC when the AMSC were exposed to synovial fluid from inflamed joints of osteoarthritis patients.

FIG. 11E illustrates results for CC19 (a/k/a C-C motif ligand 9 or MIP-3β) production by AMSC that were exposed to synovial fluid from inflamed joints of osteoarthritis patients.

FIG. 11F confirms that the data representing cytokine CX3CL1 production by AMSC exposed to synovial fluid from inflamed joints of osteoarthritis patients continues to show a significant increase, even after the data is normalized for cell number. Histograms represent means±S.D.*P<0.05

FIG. 11G confirms that the data representing cytokine CXCL6 production by AMSC exposed to synovial fluid from inflamed joints of osteoarthritis patients continue to show a significant increase, even after the data is normalized for cell number. Histograms represent means±S.D.*P<0.05

FIG. 11H confirms that the data representing cytokine CXCL1 production by AMSC exposed to synovial fluid from inflamed joints of osteoarthritis patients continue to show a significant increase even after the data is normalized for cell number. Histograms represent means±S.D.*P<0.05

FIG. 12A illustrates the effect of treating AMSC with synovial fluid from the joints of osteoarthritic patients on the production of IFNγ by the treated AMSC.

FIG. 12B illustrates the effect of treating AMSC with synovial fluid from the joints of osteoarthritic patient on the production of interleukin 8 (IL-8) by the treated AMSC.

FIG. 12C illustrates the effect of treating AMSC with synovial fluid from the joints of osteoarthritic patient on the production of interleukin 10 (IL-10) by the treated AMSC.

FIG. 13A illustrates the effect of treating macrophages with AMSC conditioned media on macrophage polarization.

FIG. 13B illustrates the effect of treating macrophages with AMSC conditioned media on macrophage polarization.

FIG. 14 illustrates the concentration of exosomes as the number of exosome vesicles per 10⁶ AMSC. The estimate was obtained by measuring the activity of acetyl-CoA acetetylcholinesterase, an enzyme present within exosomes, by the Exocet test (Exocet™ test kit, System Biosciences a/k/a SBI).

FIG. 15 illustrates the concentration of exosomes, as the number of exosomes per ml of synovial fluid, measured by Nanoparticle Tracking Analysis (Nanosight™ Malvern Instruments)

FIG. 16A illustrates that stimulation with an IFNγ/TNFα mixture induces morphological changes and inhibits proliferation of AMSCs. Representative phase contrast images (10× magnification) of AMSCs incubated with IFNγ/TNFα at concentrations of 10, 20 and 40 ng/ml for 48 hours.

FIGS. 16B illustrates the determination of cell proliferation and viability by a trypan blue exclusion assay. Y axis is fold changes with respect to untreated cells with 10 ng/ml, 20 ng/ml and 40 ng/ml IFNγ and TNFα. Columns, mean; bars, SD *significant difference from unstimulated cells; §significant difference from treatment with IFNγ/TNFα at concentration of 10 ng/ml, P<0.05

FIG. 16C illustrates the determination of cell proliferation and viability by a trypan blue exclusion assay. Y axis is percent live cells with 10 ng/ml, 20 ng/ml and 40 ng/ml IFNγ andTNFα. Columns, mean; bars, SD *significant difference from unstimulated cells; §significant difference from treatment with IFNγ/TNFα at concentration of 10 ng/ml, P<0.05

FIGS. 17A-17F illustrate the effects of stimulation with IFNγ/TNFα mixtures for inducing the expression of immunosuppressive factors, cytokines and chemokines in AMSCs. The AMSCs were treated with IFNγ/TNFα at respective concentrations of 10, 20 and 40 ng/ml for 48 h. Expression of IDO was determined by flow cytometry while PGE2 and cytokines/chemokines production was measured in supernatants by ELISA kit. Columns, mean; bars, SD, *significant difference from unstimulated cells, P<0.05.

FIG. 18A illustrates the characterization of AMSCs derived-exosomes. Immunoblotting of AMSCs-derived exosomes, Exoquick-derived supernatants (SN) and AMSCs lysate for CD9, CD63, CD81 and TSG101 exosomal protein and Calnexin, GRP94 and RISC contaminants.

FIG. 18B The concentration of exosomes was quantified as the number of exosomes ×10⁹, by measuring the enzymatic activity of the exosomal AChE enzyme with the Exocet kit. Columns, mean; bars.

FIG. 18C illustrates a representative graph of frequency size distribution is shown.

FIG. 18D Particles sizes were quantified by qNano system. Columns, mean; bars.

FIG. 19A Representative phase contrast microscopic images (20× magnification) of monocytes differentiated into macrophages in presence of GM-CSF alone (CTRL) or in combination with exosomes isolated from the supernatants of unstimulated (EXO UNSTIM) or cytokines-activated (EXO IFNγ/TNFα 10 , 20 and 40 ng/ml) AMSCs. The circles evidence cells with elongated, spindle-like morphology, a typical feature of M2 macrophages.

FIG. 19B Flow cytometry analysis of cell surface molecules CD163 on macrophages.

FIG. 19C Flow cytometry analysis of cell surface molecules CD206 on macrophages.

FIG. 19D Flow cytometry analysis of cell surface molecules CD80 on macrophages. The levels of expression are presented as median fluorescent intensity (MFI) fold change respect untreated cells. Columns, mean; bars, SD, *significant difference from unstimulated cells, P<0.05.

FIG. 20A Illustrates exosomes derived from AMSCs pre-activated with inflammatory cytokines contained miRNA involved in M2 macrophages polarization. The concentration of miR-34 was measured in exosomes produced by AMSCs treated with or without 10, 20 and 40 ng/ml IFNγ/TNFα by qRT-PCR. Columns, mean; bars, SD, *significant difference from exosomes of unstimulated cells, P<0.05. (G) Monocytes were differentiated in macrophages with GM-CSF in presence of exosomes isolated from the supernatants of unstimulated (EXO UNSTIM) or cytokines-activated (EXO IFNγ/TNFα 10, 20 and 40 ng/ml) AMSCs. Cell lysates were subjected to Western blot analysis with specific antibody against to IRAK1, Notch1, Sirp-β1 and β-actin.

FIG. 20B As for FIG. 20A but with miR-127.

FIG. 20C As for FIG. 20A but with miR-21

FIG. 20D As for FIG. 20A but with miR-135.

FIG. 20E As for FIG. 20A but with,miR-146.

FIG. 20F As for FIG. 20A but with miR-155.

FIG. 21A Illustrates immunophenotype of AMSCs with a representative flow cytometry histogram of AMSCs stained for mesenchymal stem cell marker CD29. The antibodies (white column) were compared with their appropriate isotype control (grey column)

FIG. 21B As for FIG. 21A but with CD73.

FIG. 21C As for FIG. 21A but with CD90.

FIG. 21D As for FIG. 21A but with CD105.

FIG. 21E As for FIG. 21A but with hematopoietic marker CD34

FIG. 21F As for FIG. 21A but with hematopoietic marker CD45.

FIG. 22A Confirms that cytokines contamination from the culture medium did not affect macrophages polarization. Monocytes were differentiated into macrophages in presence of GM-CSF alone (CTRL) or in combination with cytokines contaminants isolated by polymer precipitation method from the culture medium supplemented with IFNγ/TNFα at different concentration (10, 20 and 40 ng/ml). Flow cytometry analysis of cell surface molecule CD80 was measured. The levels of expression are presented as median fluorescent intensity (MFI) fold change respect untreated cells

FIG. 22B As for FIG. 22A but flow cytometry analysis of cell surface molecule CD163 was measured.

Monocytes were differentiated into macrophages in presence of GM-CSF alone (CTRL) or in combination with cytokines contaminants isolated by polymer precipitation method from the culture medium supplemented with IFNγ/TNFα at different concentration (10, 20 and 40 ng/ml). Flow cytometry analysis of cell surface molecules CD80 (A) and CD163 (B) on macrophages.

### DETAILED DESCRIPTION OF THE INVENTION

The present invention provides anti-inflammatory exosomes and methods of obtaining and using anti-inflammatory exosomes to inhibit or downregulate the immune system inflammatory response in a subject. The subject is broadly any animal, including a mammal and/or avian, and in particular embodiments the mammal is a human or veterinary patient in need of treatment thereof.

The invention also provides immunotherapy employing the inventive anti-inflammatory exosomes for treating or preventing cancer, or a precancerous condition, in a subject by downregulating or inhibiting inflammatory processes that drive certain cancers or precancerous conditions.

In order to appreciate the present invention, the following terms are defined. Unless otherwise indicated, the terms listed below will be used and are intended to be defined as stated, unless otherwise indicated. Definitions for other terms can occur throughout the specification. It is intended that all singular terms also encompass the plural, active tense and past tense forms of a term, unless otherwise indicated.

Anti-inflammatory exosomes according to the invention are exosomes that when administered to a subject, such as a mammal, having an inflammatory disease or disorder, will inhibit or downregulate the inflammatory process. The anti-inflammatory exosomes are produced from adult stem cells that have been activated to enhance the immunosuppressive activity of exosomes produced or secreted by those adult stem cells. The process broadly includes contacting, e.g., culturing, suitable mammalian adult stem cells with an appropriate activating composition. Once contacted with the activating composition, the treated adult stem cells release anti-inflammatory exosomes that, when collected, purified and administered to a subject diagnosed with an inflammatory disease or disorder, will inhibit or downregulate inflammation in the treated subject.

The term, adult stem cells, as used herein, is intended to include stem cells derived from the tissues, blood or body fluids of a non-embryonic and non-fetal animal, such as a mammal. This definition includes stem cells derived from mammalian umbilical cord blood or tissue, and/or mammalian placental blood or tissue, unless otherwise specified.

The phrase “consisting essentially of” means that the composition or method may include additional ingredients and/or steps, but only if the additional ingredients and/or steps do not materially alter the basic and novel characteristics of the claimed composition or method, i.e., the additional ingredient and/or step(s) would serve no purpose material to the claimed composition or method.

In certain embodiments of the invention, the adult stem cells are derived, without limitation, from umbilical cord, placenta, non-fetal cells found in amniotic fluid, adipose tissue, bone marrow, peripheral blood, hair follicles, the gastrointestinal organs, nervous system, i.e., central and/or peripheral nervous system, circulatory system, respiratory system, the immune system, and secretory organs such as the mammary glands.

Adult stem cells derived from gastrointestinal organs include, without limitation, adult stem cells derived from the mucosal surface, myenteric plexus, smooth muscle and/or glandular tissues of the esophagus, stomach, small intestine, large intestine, liver, pancreas, gall bladder, salivary glands, and other gastrointestinal storage and/or secretory organs.

Adult stem cells derived from nervous system tissue, include those derived from the central nervous system, including the brain, retinas, and spinal cord. Adult stem cells derived from nervous system tissue also include those derived from the peripheral nervous system.

Adult stem cells derived from the circulatory system include those derived from blood cells, as well as those derived from the heart, e.g., heart muscle and/or heart valves, arteries, veins, and lymphatic system.

Adult stem cells derived from the respiratory system include those derived from the lungs, bronchi, bronchioles, pharynx and nasopharynx.

Adult stem cells derived from the immune system include those adult stem cells associated with the immune system that are derived from the bone marrow, spleen and peripheral tissues.

In particular embodiments, adult stem cells include, for example, adipose derived mesenchymal stem cells (AMSC), bone marrow, or umbilical cord derived hematopoietic stem cells, bone marrow derived endothelial stem cells, olfactory derived neural crest stem cells, mammary derived stem cells, and/or intestinal derived stem cells.

Optionally, the adult stem cells are derived from the subject to be treated.

The term “culturing” refers to the in vitro maintenance, differentiation, and/or propagation of cells in suitable media. By “enriched” is meant a composition comprising cells present in a greater percentage of total cells than is found in the tissues where they are present in an organism.

Methods for isolating adult stem cells include methods known to the art. Methods of isolating and expanding adipose-derived stem cells are described, for example, Bunnell B A et al., 2008, Methods. 45(2):115-20. doi: 10.1016/j.ymeth. 2008.03.006. Methods for isolating and expanding mesenchymal stem cells/multipotential stromal cells from human bone marrow are described, for example, by, Penfornis P. et al., 2011 Methods Mol Biol.: 698:11-21. doi: 10.1007/978-1-60761-999-4_2. Methods for isolating, expanding and characterizing mesenchymal stem cells from human bone marrow, adipose tissue, umbilical cord blood and matrix are also described, for example, by Secunda R, 2015, Cytotechnology. 67(5):793-807. doi: 10.1007/s10616-014-9718-z, and treating the harvested adipose tissue as described by Example 10, below.

Broadly, the “activating composition” according to the invention is any composition that is effective to induce a cultured adult stem cell to secrete anti-inflammatory exosomes. In particular embodiments, the activating composition includes one or more of the following:

(i) fluid extracted from inflamed tissue, or other anatomical structure, of a mammal diagnosed with an inflammatory disease or disorder,

(ii) blood, plasma or serum from a mammal diagnosed with an inflammatory disease or disorder,

(iii) at least one cytokine at a concentration sufficient to enhance the effectiveness of the activating composition comprising (i) or (ii), or

(iv) at least one cytokine at a concentration sufficient to induce the adult stem cells to secrete anti-inflammatory exosomes capable of inhibiting inflammation in a mammal, and the activating composition excludes the fluid of (i) and/or the blood, plasma or serum of (ii).

Preferably, the at least one cytokine of (iii) or (iv) is selected from the group consisting of interferon-gamma (IFNγ), interleukin-1α (IL-1α), interleukin-1-β (1IL-1β), interleukin-6 (IL-6), interleukin 12b (IL-12b), tumor necrosis factor-α (TNFα) and combinations thereof, and is present in a concentration ranging from about 10 ng/ml to about 50 ng/ml. Optionally, the cytokine of (iii) and/or (iv) is from an exogenous source.

An “exogenous cytokine” is a cytokine that is added from a source outside the culture medium and that added to the culture medium to a level or concentration above that which is found in the fluid, blood, plasma or serum obtained from the subject.

The embodiment of (iv) provides for a synthetic activating composition that includes one or more cytokines, and optionally other agents, that induce the cultured adult stem cells to secrete anti-inflammatory exosomes while excluding the fluid, blood, serum and/or plasma obtained from a subject having an inflammatory condition. Optionally, the synthetic activating composition is prepared in the form of liposomes designed to mimic the properties and composition of exosomes, preferably ranging in size from about 40 nm to about 100 nm, with a density between 1.15 g/ml (Lane et al., 2015, Scientific Reports 5, Article number: 7639 doi:10.1038/srep07639).

The synthetic activating composition includes, without limitation, cytokines, such as interferon gamma (IFNγ), tumor necrosis factor alpha (TNFα), interleukin 1 alpha and beta (IL1α and IL1β), in concentrations ranging from about 10 ng/ml to about 50 ng/ml.

In other embodiments of the invention, the cultured adult stem cell may be genetically engineered to express a gene or genes encoding one or more heterologous activating agents. Such genes would encode cytokines, including IFNγ, TNFα, IL1α and/or IL1β. Alternatively, the cultured adult stem cell may be grown on a substrate of supporting cells, such as fibroblasts, engineered to express the activating agents listed above.

Culture media for culturing mammalian cell lines, including adult stem cells, in vitro are known to those skilled in the art and commonly used. For example, a suitable culture medium is Eagle's minimal essential medium with 10% Fetal Bovine Serum, 10 mL/L Pen/Strep Solution, 2 mM Ala-Gln solution, 10 ng/ml Epidermal Growth Factor, 10 μg/ml Insulin solution, 100 μM 2-fosfo-L-ascorbic acid trisodium salt, and 0.01 μM Dexamethasone.

Adult stem cells are cultured, for example, by inoculating culture medium, with from about 30,000 to about 50,000 cells per ml.

After incubating for from about 2 to about 4 days at 37° C., the inoculated culture medium is collected and the exosomes purified and isolated from the culture medium. This can be accomplished by any suitable art-known method. For example, see Robbins et al., US20060116321 or Lane et al., Id., Brownlee, et al., 2014, J Immunol Methods, 407: 120-126. doi: 10.1016/j.jim.2014.04.003. These methods include, for example, the original method of separating exosomes by differential ultracentrifugation, and newer methods, such as polymer precipitation (ExoQuick™ from SBI, Palo Alto, Calif.), immunoaffinity capture (Greening et al. 2015, Methods in Molecular Biology, Impact Factor: 1.29), immune magnetic capture (Exo-FLOW™, SBI), the Invitrogen Total Exosome Isolation Kit (Life Technologies, USA) and the ExoSpin Exosome Purification Kit (Cell Guidance Systems, USA).

Immuno-affinity purification is a method to selectively capture specific exosomes based upon surface markers. This approach employs magnetic beads covalently coated with streptavidin, which can be coupled in high affinity fashion with biotinylated capture antibody. Captured exosomes are eluted and are intact and bioactive.

Purified exosomes are quantified by determining the protein content and the activity of acetyl-CoA acetetylcholinesterase, and are analyzed for size distribution and concentration by nanoparticle tracking analysis. Isolated exosomes are validated for exosomal marker expression by flow cytometry and Western blot.

The invention also provides methods of treating subjects, including mammalian subjects, suffering from diseases or disorders caused by, or exacerbated by, inflammatory disorders and/or requiring modulation of the immune system. When the mammalian subject is a human subject, such diseases or disorders are contemplated to include, without limitation, arthritis, allergy, asthma, or an autoimmune disease such as, rheumatoid arthritis, osteoarthritis, juvenile rheumatoid arthritis, systemic lupus erythematosis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo. Other conditions which may desirably be treated include diseases such as muscular dystrophy, and conditions in which inflammation can interfere with proper healing, such as an accidental or iatrogenic wound in soft tissue, ligament, or bone, or tissue damaged by a non-immune event, for example, heart muscle following myocardial infarction.

The diseases or disorders contemplated to be treated according to the invention include both systemic and tissue specific diseases or disorders. Systemic diseases include, for example, the various manifestations of metabolic syndrome, such as coronary artery disease, e.g., atherosclerosis and ischemic heart disease, type 2 diabetes, diseases of other end artery organs, peripheral artery disease and related conditions.

Tissue specific diseases include inflammatory diseases confined to a particular organ or tissue type, as follows.

Diseases or disorders of the respiratory system to be treated include, for example, asthma, bronchitis and chronic obstructive pulmonary disease (COPD). Diseases or disorders of the gastrointestinal system to be treated include, for example, inflammatory bowel disease or IBD, such as ulcerative colitis and Crohn's disease. Skin diseases to be treated include, for example, dermatitis, eczema and psoriasis. Diseases or disorders of the central nervous system to be treated include, for example, Alzheimer's disease, Parkinson's disease and optionally migraine conditions. Diseases or disorders of the musculature to be treated include, for example, polymyositis, dermatomyositis, inclusion body myositis (IBM) and juvenile myositis. Diseases or disorders of the joints to be treated include, for example, rheumatoid arthritis, osteoarthritis, amyloidosis, ankylosing spondylitis, bursitis, psoriatic arthritis, Still's disease and others.

In certain embodiments, the inflammatory diseases or disorders are those predisposing to a higher risk of cancer, such as, for example, the various inflammatory gastrointestinal diseases, such as inflammatory bowel disease and Barrett's esophagous; chronic bacterial infections, e.g., infection with H. pylori, chronic asbestosis, silicosis and other tissue inflammations caused by inhaling or ingesting non-biodegradable dusts, infections with parasites such as Schistosomiasis, infections with viruses, such as the Epstein-Barr virus, human papilloma virus, hepatitis B virus, and human herpes virus-8, chronic inflammation induced by exposure to tobacco products and so forth.

In another embodiment, the inflammatory disease or disorder to be treated is osteoarthritis, and the activating composition includes, without limitation, synovial fluid from one or more inflamed joints of the osteoarthritic mammal The mammalian subject can be a human subject, or a veterinary subject, such as, for example, and without limitation, domesticated animals, animals typically kept as pets or work animals, and or exotic animals, e.g., zoo animals, for which it is desired to treat an inflammatory disorder. For example, it is contemplated that the inventive methods be applied, without limitation to subjects that include humans and veterinary subjects. Veterinary subjects include mammals and avians. Mammalian subjects include, simply by way of example, non-human primates, bovine (e.g., cattle or dairy cows), porcine (e.g., hogs or pigs), ovine (e.g., goats or sheep), equine (e.g., horses), canine (e.g., dogs), feline (e.g., house cats), camels, deer, antelopes, rabbits, guinea pigs and rodents (e.g., squirrels, rats, mice, gerbils, and hamsters), cetaceans (whales, dolphins, porpoise), pinnipeds (seals, walrus). Potential avian subjects include, simply by way of example, Anatidae (e.g., swans, ducks and geese), Columbidae (e.g., doves and pigeons), Phasianidae (e.g., partridges, grouse and turkeys) Thesienidae (e.g., domestic chickens), Psittacines (e.g., parakeets, macaws, and parrots), game birds, and ratites, (e.g., ostriches).

The invention also provides purified anti-inflammatory exosomes prepared by

(a) culturing adipose derived mesenchymal stem cells (AMSC) in a suitable culture medium, the culture medium comprising fluid extracted from inflamed tissue of a mammal diagnosed with an inflammatory disease or disorder, until the culture medium is conditioned,

(b) collecting the conditioned culture medium from (a),

(c) isolating AMSC exosomes from the conditioned culture medium by polymer precipitation, and/or by any other art known method.

Without meaning to be bound by any theory or hypothesis as to how the invention operates, exosomes produced by stem cells cultured in the presence of an activating composition, which includes factors secreted from inflammatory tissue, induce macrophages present in inflamed tissues to change from an M1 pro-inflammatory phenotype to the M2 macrophage immunosuppressive phenotype.

M1 macrophages are pro-inflammatory cells with potent anti-microbial activity that promote T helper cell responses. M1 macrophages have also been implicated in many inflammatory disease, such as osteoarthritis. M2 macrophages are immunosuppressive cells that can support T helper cell 2 (Th cell 2) associated effector functions. M2 macrophages, produce anti-inflammatory cytokines (Röszer T, 2015, Mediators Inflamm. 2015:816460. doi: 10.1155/2015/816460), and are thought to play a major role in the resolution of inflammation, tissue remodeling and in wound repair.

Thus, the invention provides for methods of treating a disease or disorder caused by, or exacerbated by, an inflammatory condition. The anti-inflammatory exosomes are administered by any clinically appropriate route to deliver the exosomes to the inflamed tissue or organ, or may be delivered systemically when clinically appropriate. By way of example, the anti-inflammatory exosomes are administered by a route such as, intravenously, intramuscularly, intraarticularly, subcutaneously and/or intrathecally and/or by direct injection, infusion or instillation, intranasally or by inhalation, into an inflamed tissue or organ, as well as topically to the skin.

An “effective amount” is an amount sufficient to effect beneficial or desired results, such as a downregulated inflammatory response, treatment, prevention or amelioration of a medical condition (disease, infection, etc.). An effective amount can be administered in one or more administrations, applications or dosages. The effective amount, i.e., a suitable dosage, will vary depending on body weight, age, health, disease or condition to be treated and route of administration. The dose of exosomes administered to a subject is in an amount effective to achieve the desired beneficial therapeutic response in the subject over time.

The artisan will be readily able to determine the amount of exosomes to be administered by titrating the dose and duration of administration to reach an optimal clinical response, such as a reduction in the inflammatory process of the disease or disorder that is being treated.

In particular, the anti-inflammatory exosomes are administered systemically, in an amount ranging from about 1.5×10¹⁰ to about 1.5×10¹³ exosome particles per kilogram of total body weight. Aternatively, the anti-inflammatory exosomes are administered in an amount ranging from about 1.5×10¹⁰ and 1.5×10¹¹ exosome particles injected or infused into a localized tissue or anatomical space.

The number of exosomes in a preparation can be determined by any art known method. In a non-limiting example, exosome particle numbers can be determined by direct counting using a NanoSight instrument, such as a NanoSight® NS300, NanoSight NS500® or NanoSight® LM10 (Malvern Instruments, Ltd, Worcestershire, UK). Alternatively, the number of exosomes can be estimated by measuring the activity of Acetyl-CoA Acetetylcholinesterase, an enzyme present within exosomes, and then estimating the exosome count by reference to a pre-prepared standard curve of exosome counts verses Acetyl Co-A levels.

The treatment is repeated as needed until a positive anti-inflammatory result is obtained. Optionally, the treatment is repeated at a daily, weekly or monthly interval, as needed, in order to maintain suppression of the inflammatory process.

In a further embodiment, the invention contemplates co-treating a mammal in need thereof, with at least one additional anti-inflammatory agent, the at least one additional anti-inflammatory agent including, for example, a steroidal anti-inflammatory, a non-steroidal anti-inflammatory, an anti-TNF alpha antibody and combinations thereof.

Steroidal anti-inflammatory medications include, without limitation, cortisone, triamcinolone, dexamethasone, hydrocortisone, prednisone, methylprednisolone, prednisolone hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Steroidal anti-inflammatory medications formulated for inhalation therapy include, without limitation, beclomethasone, budesonide, ciclesonide, flunisolide, fluticasone, and triamcinolone.

Non-steroidal anti-inflammatory drugs (NSAIDs) represent a large group of therapeutic agents with analgesic, anti-inflammatory, and anti-pyretic properties. NSAIDs typically reduce inflammation by blocking the cyclooxygenase 1 and/or cyclooxygenase 2 (COX 1 and COX 2) enzymes. NSAIDs that selectively inhibit COX 2 enzymes are more sparing of the gastric mucosa, where COX1 is predominant Representative NSAIDs include, without limitation.

Non-steroidal anti-inflammatory drugs (NSAIDs) represent a large group of therapeutic agents with analgesic, anti-inflammatory, and anti-pyretic properties. NSAIDs typically reduce inflammation by blocking the cyclooxygenase 1 and/or cyclooxygenase 2 (COX 1 and COX 2) enzymes. NSAIDs that selectively inhibit COX 2 enzymes are more sparing of the gastric mucosa, where COX1 is predominant Representative NSAIDs include, without limitation, aceclofenac, acemetacin, actarit, alcofenac, alminoprofen, amfenac, aloxipirin, aspirin, azapropazone, benzydamine (prostaglandin synthase inhibitor), butibufen, celecoxib, chlorthenoxacin, choline salicylate, dexketoprofen, diclofenac, diflunisal, emorfazone, epirizole; etodolac, etoricoxib, feclobuzone, felbinac, fenbufen, fenclofenac, fenoprofen, flurbiprofen, glafenine, hydroxylethyl salicylate, ibuprofen, indomethacin, indoprofen, ketoprofen, ketorolac, loxoprofen, lumiracoxib, mefenamic acid, meloxicam, metamizole, mefenamic acid, metiazinic acid, mofebutazone, mofezolac, nabumetone, naproxen, nifenazone, niflumic acid, oxaprozin, piroxicam, pranoprofen, propyphenazone, proquazone, protizinic acid, rofecoxib, salicylamide, salsalate, sulindac, suprofen, tiaramide, tinoridine, tolfenamic acid, tolmetin, and valdecoxib.

Antibody based anti-inflammatory medications include, without limitation, infliximab, etanercept, alemtuzumab, adalimumab, omalizumab, efalizumab, alefacept, natalizumab, abatacept, certolizumab pegol, golimumab, canakinumab, tocilizumab, ustekinumab (MAbs. 2010 May-June; 2(3): 233-255), Vedolizumab, talizumab, abrilumab, inclacumab, anifrolumab, anrukinzumab, benralizumab, brodalumab, clazakizumab, clenoliximab, eldelumab, etrolizumab, gomiliximab, mavrilimumab, oxelumab, pateclizumab, perakizumab, quilizumab, rontalizumab, sirukumab, tezepelumab, Tildrakizumab, and zanolimumab.

### EXAMPLES

The following examples are provided in order to illustrate the present invention, without intending to limit the scope of the present invention.

### Example 1

### Isolation of Exosomes from Synovial Fluid by Polymer Precipitation

Exosomes were isolated by polymer precipitation (ExoQuick™ from SBI, Palo Alto, Calif.). This technology captures and collects exosomes in “polymer nets,” that are recovered by a low speed centrifugation. Once the exosome pellet was obtained, the supernatant containing excess polymer was removed and the pelleted exosomes were then re-suspended in phosphate-buffered saline (PBS) solution, dissolving the polymer net and releasing intact exosomes.

### Example 2

### Validating Isolated Exosomes

In order to confirm that functional exosomes were isolated by the polymer precipitation process of Example 1, the identity of the exosomes was validated by measuring the presence of appropriate biomarkers, as follows.

Exosomes were isolated from the synovial fluids of patients diagnosed with osteoarthritis (n=6) by the method described above, and the exosomes validated by Western blot, using antibodies targeting specific exosomal markers: tetraspanins, CD9, CD63, CD81 (localized to the exosomal surface) and the protein expressed by tumor susceptibility gene 101 (“TSG101”) that is found inside exosomes.

Isolated exosomes were lysed in RIPA buffer (150 mM sodium chloride, 1% NP-40, 0.5% sodium deoxycholate, 0.1% SDS, 50 mM Tris, pH 8.0) supplemented with protease inhibitors (Sigma-Aldrich) for 30 minutes on ice. The lysate was quantified for Bradford assay (Bio-Rad) and 25 μg of proteins were mixed with 4× sample buffer (8% SDS, 20% 2-mercaptoethanol, 40% glycerol, 0.008% bromophenol blue, 0.25M Tris, pH 6.8) and boiled for 10 minutes at 95° C. Proteins were resolved by SDS-PAGE, transferred to PVDF membranes, blocked in 5% non-fat powdered milk or BSA in TBS-T (20 mM Tris pH 7.5, 150 mM NaCl, 0.1% Tween-20) and probed with the following anti-human antibodies: anti-CD9 (1:1000, SBI), anti-CD63 (1:1000, LS Bio), anti-CD81 (1:500, Abcam), anti-TSG101 (1:500, Abcam). Secondary antibodies conjugated to horseradish peroxidase (1:1000, Dako) visualized the proteins by way chemilluminescence (ECL Western blotting substrate, Thermo Scientific).

In addition, in order to further validate the exosomes by flow cytometry, exosomes were bound to latex beads that were conjugated with anti-CD63 antibody, thus avoiding any non-specific binding of the beads that might result from their small size. The expression of CD81 and CD9 by the exosomes was confirmed by cytofluorimetric data, where CD7 was used as a negative control to demonstrate the specificity of the assay. The primary antibodies used were: anti- CD9 Alexa 647 (Serotec), anti-CD81 FITC (Biolegend) and anti-CD7 PE (Becton Dickinson) or isotype control (BD Biosciences). Exosomes were analyzed using a FACSCalibur flow cytometer (BD Biosciences).

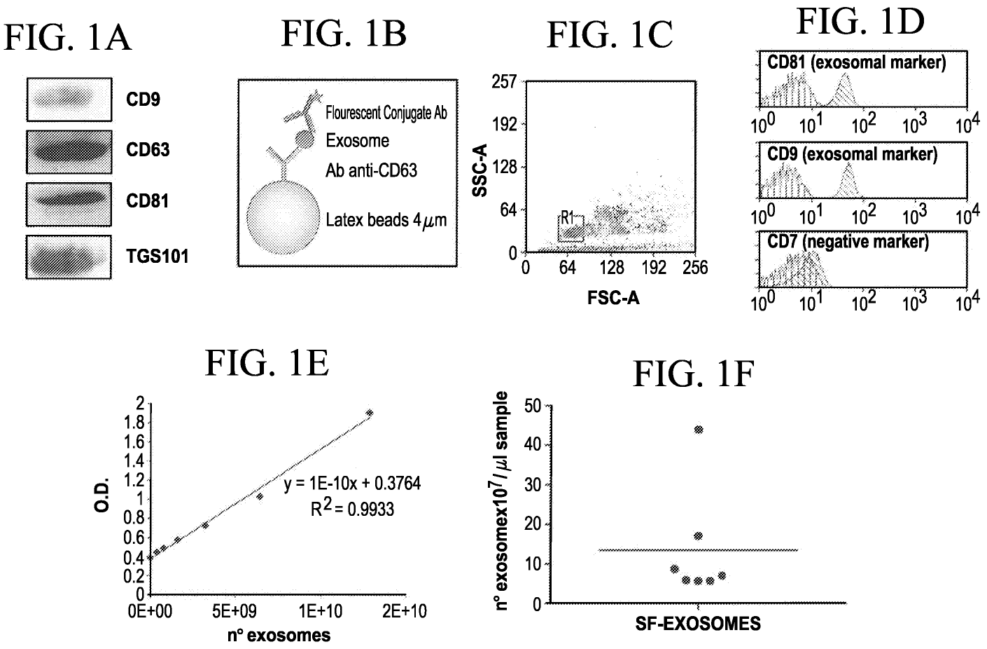

The obtained exosomes were quantified by determining the activity of acetyl-CoA esterase (“AChE,” Exocet™ test kit, SBI). AChE is an enzyme known to be found within all exosomes tested to date. A standard curve, as measured by NanoSight® analysis, that was calibrated to exosome numbers, was included in the Exocet kit. This is illustrated by FIGS. 1E and 1F.

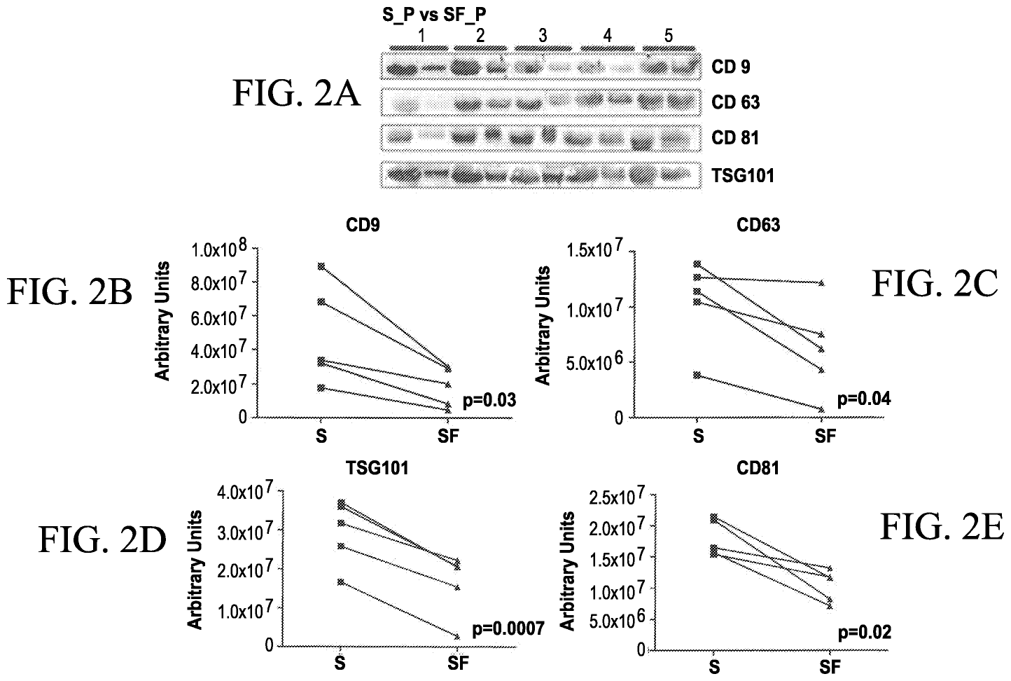

Because it was difficult to obtain comparison synovial fluid from healthy volunteers, exosomes isolated by polymer precipitation from synovial fluid were compared to exosomes isolated from serum of the same osteoarthritis patients (n=5). As illustrated by FIGS. 2A through 2E, the expression of the main exosomal markers, evaluated by Western blot, was significantly lower in synovial fluid exosomes, relative to the respective corresponding serum exosome counterpart from the same patient, suggesting a lower content of exosomes in synovial fluid. Thus, based on the results obtained by Western blot, it was observed that the synovial fluid has a lower content of exosomes than the serum of the same patient.

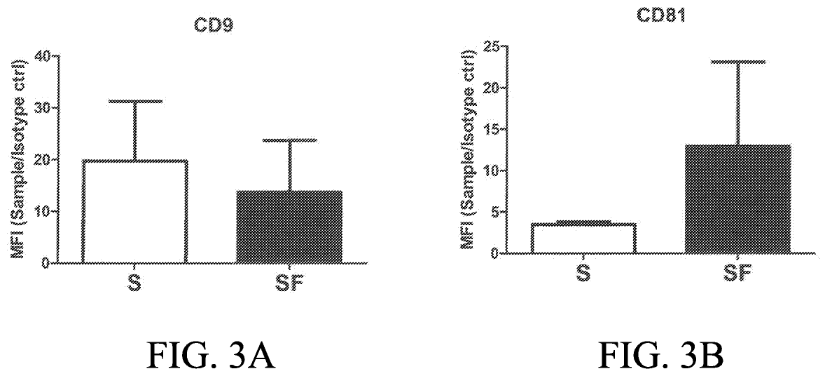

The same comparison between exosomes obtained from patient synovial fluid and respective patient serum, was also conducted by flow cytometry, by the method described above, as illustrated by FIGS. 3A and 3B. The cytofluorimetric data showed that the synovial fluid-derived exosomes (SF) have a higher expression of CD81 as compared to serum-derived exosome (S) isolated from the same patient, while no difference was observed for the expression of CD9. Histograms represent means±S.D.

Without meaning to be bound by any theory or hypothesis as to the operation of the invention, the above results suggest that exosomes in synovial fluid have different characteristics than exosomes present in serum.

### Example 3

### Effects of Exosomes Isolated from Synovial Fluid on M1 Macrophages

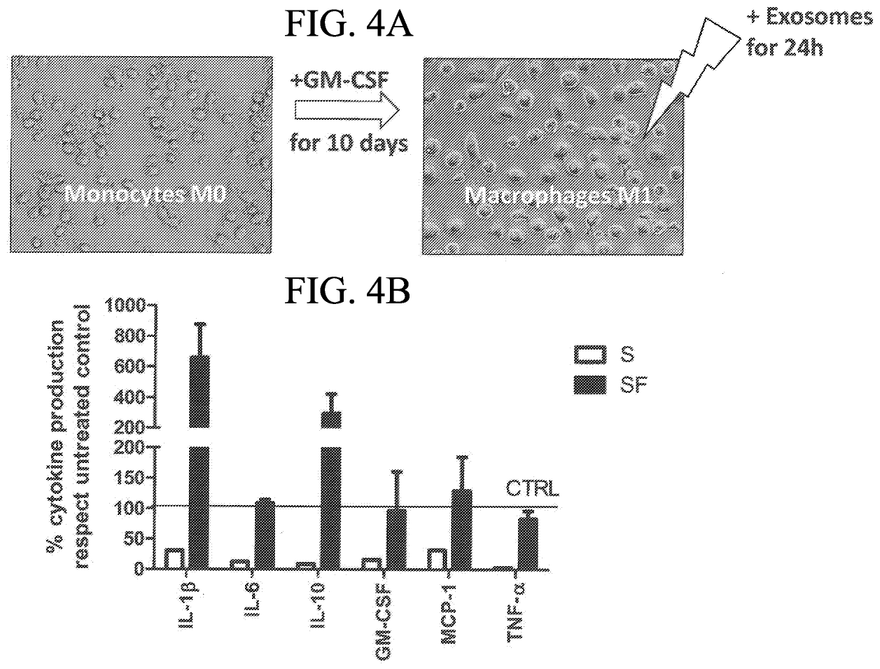

In order to verify the immune regulatory properties of synovial fluid-derived exosomes, exosomes were isolated by polymer precipitation as described by Example 1. CD14+ monocytes were purified from healthy donor peripheral blood mononuclear cells (“PBMC”) using a human CD14+ cell enrichment kit (StemCell Technologies), differentiated into M1 macrophages by incubation with 100 ng/ml granulocyte macrophage-colony stimulating factor (GM-CSF, Peprotech) for 10 days, and then stimulated with 150 UI/ml interferon (“IFN”) gamma (“γ”) (Peprotech) and 10 ng/ml lipopolysaccharide (LPS) (Sigma-Aldrich) in the presence of exosomes for 24 h, as illustrated by FIG. 4A.

M1 macrophages are pro-inflammatory cells with potent anti-microbial activity that promote T helper cell responses. Cytokine production in M1 macrophages was measured by Bio-Plex® Assay (Bio-Rad Laboratories) conducted with supernatants derived from the M1-macrophages. The resulting data (FIG. 4B) shows that serum (S)-derived exosomes inhibit cytokine secretion in LPS/IFNγ stimulated M1 macrophages, while synovial fluid (SF)-derived exosomes significantly increase the release of IL-1β and IL-10 in the same cells. Controls were exosome-untreated, LPS/IFNγ stimulated M1 macrophages. See FIGS. 4A and 4B. In FIG. 4B, the histograms represent mean±S.D.

### Example 4

### Effects of Exosomes Isolated from Synovial Fluid on Cytokine Mrna Expression in M1 Macrophages

The M1-stimulatory properties of synovial fluid-derived exosomes isolated by polymer precipitation (Example 1) from osteoarthritic patients, was confirmed by cytokine gene expression analysis. M1 macrophages were incubated with synovial fluid (SF) or synovial fluid-derived exosomes (EXO-SF) for 6 h and expression of mRNA encoding for IL-1β, IL-6, TNF-α and IL-12b, respectively, was measured by RT-PCR. LPS was used as positive control for macrophages stimulation.

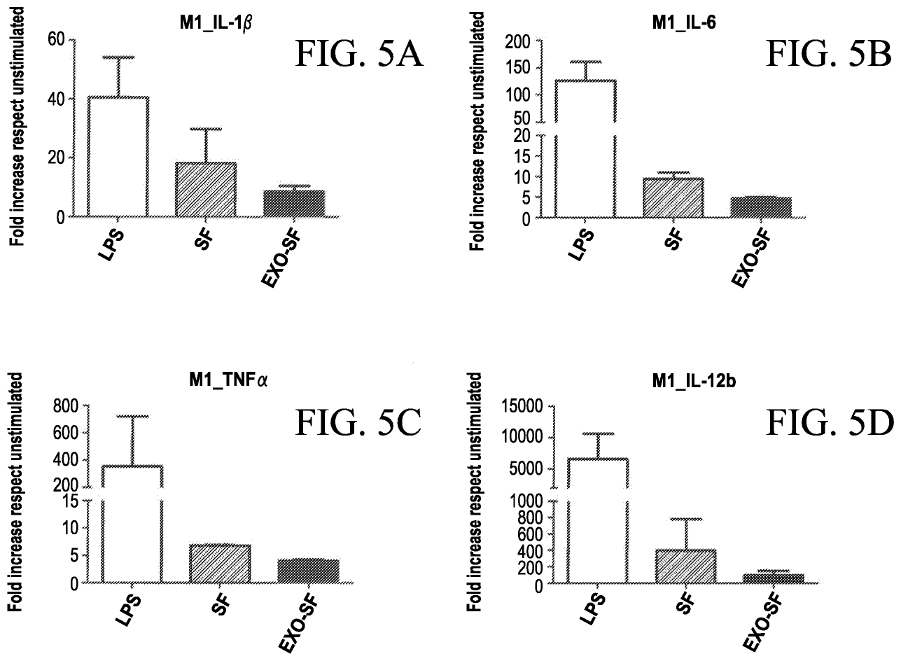

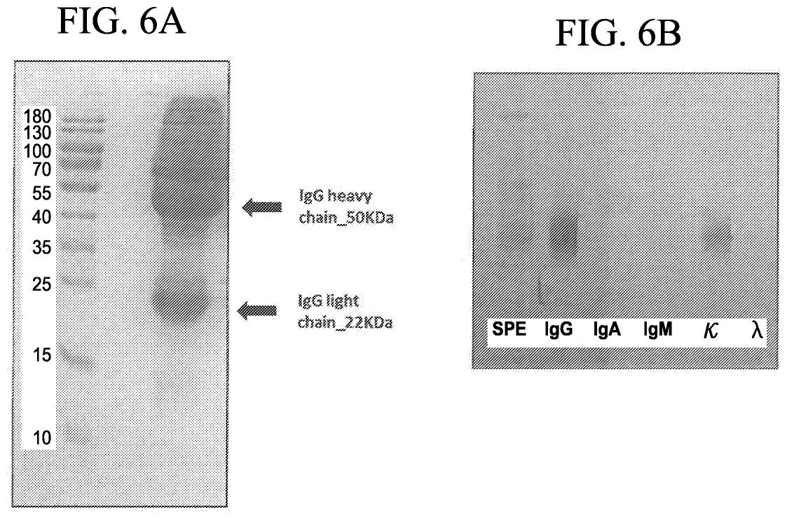

The results (FIGS. 5A through 5D) show that the transcriptional level of mRNA encoding for IL-1β, IL-6, TNF-α and IL-12b was increased after both treatments, indicating that the pro-inflammatory property of synovial fluid was maintained in the respective exosomal fractions. However, it was observed that the exosomal fraction stimulated M1 less than synovial fluid in toto. Histograms represent means±S.D.

The above data shows that SF-derived exosomes are able to stimulate the production of pro-inflammatory cytokines.

### Example 5

### Immune Complex Contamination in Exosomes Purified from Synovial Fluid by Polymer Precipitation

The presence of immune-complexes in the above described synovial fluid exosome samples was evaluated by electrophoresis with Coommasie gel staining and immunofixation. The presence of immune complexes was confirmed as illustrated by FIGS. 5A and 5B. Thus, with polymer precipitation, immune complexes co-precipitate with exosomes, and the co-precipitated immune complexes may have had functional effects on the above described test results.

### Example 6

### Exosome Purification by Immunocapture with Magnetic Beads (Exo-Flow)

To overcome the problem of immune-complex contaminants and other possible contamination, exosome samples prepared using the method of Example 1 were further purified using immunological separation.

Isolated exosomes obtained from the method of Example 1 were bound to magnetic beads (Exo-FLOW™, SBI). Magnetic beads [9.1 μm, 1.6×107 beads/ml] were coupled with anti-CD9 or anti-CD63 or anti-CD81 biotinylated antibody for 2 h on ice, and then incubated with exosomes on a rotating rack at 4° C. overnight for capture. The beads were coated with the three different antibodies separately and then mixed for the capture of exosomes

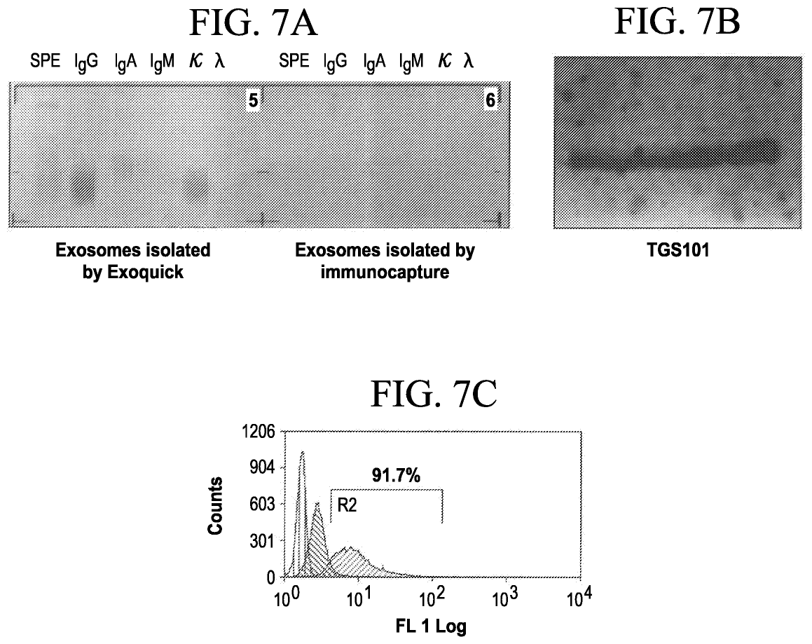

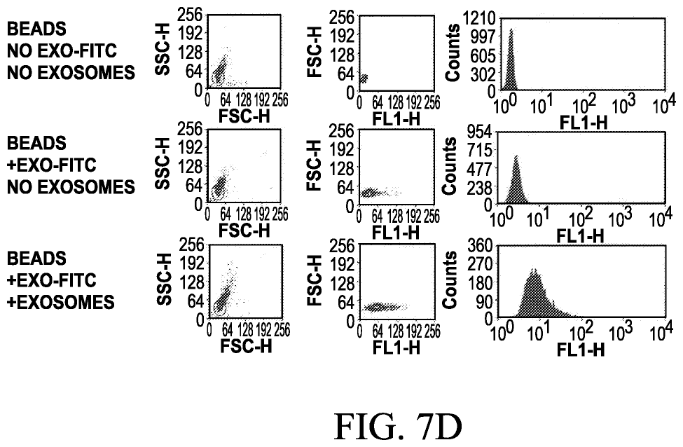

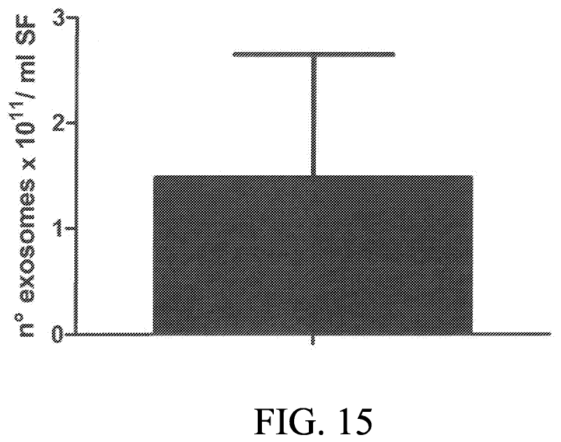

To verify the specificity of the purification by immunocapture, the absence of immune-complexes was confirmed by immunofixation, as illustrated by FIGS. 7A-7D. Exosomes were validated by Western blot using the specific exosomal marker TSG101 and by flow cytometry using Exo-FITC™ staining. This staining takes advantage of the finding that most exosome surface proteins have modifications, such as, glycosylations, carbohydrate additions, etc. that are bound by the protein component of SBI's protein-fluorescein isothiocyanate (FITC) conjugate, commercially available as Exo-FITC™ (SBI). The data indicate that about 90% of the exosomes bound to the beads were positive for the staining The SF-derived exosomes were analyzed for size distribution and concentration by NanosightTM, and the results shown by FIG. 15.

### Example 7

### Effect of Purified Exosomes Isolated from Synovial Fluid on M1 Macrophage Cytokine Gene Expression by RT-PCR

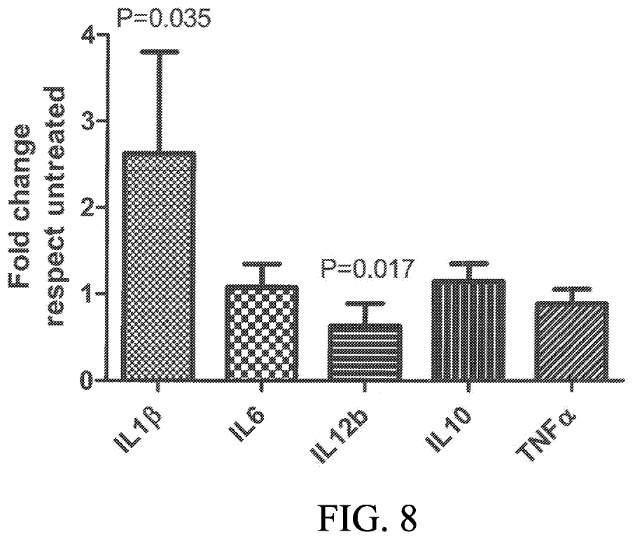

The M1-stimulatory properties of synovial fluid-derived exosomes purified by immunecapture was evaluated by cytokine gene expression analysis. M1 macrophages were incubated with synovial fluid-derived exosomes for 6 h and cytokine coding mRNA expression was evaluated by RT-PCR. A significant upregulation in gene expression of IL-β was observed, together with a down regulation of the expression of IL12b. This is illustrated by FIG. 8. In FIG. 8, the histograms represent means±S.D.

The overall results confirm that synovial fluid-derived exosomes are able to stimulate M1 macrophages. M1 macrophages have been implicated in mediating articular destruction in affected joints (Kennedy et al., 2011, Front Immunol. Vol. 2, Article 52, pages 1-9).

### Example 8

### Effect on AMSC of Synovial Fluid from Inflamed Joints

In order to evaluate the effects of synovial fluid from inflamed joints on AMSC, the stem cells were isolated from the adipose tissue of a human subject, e.g., by the method of Secunda, Id. The adipose cells are cultured, then isolated, according to the method of Bunnel et al., 2008 Methods (San Diego, Calif).45(2):115-120. doi:10.1016/j.ymeth. 2008.03.006).

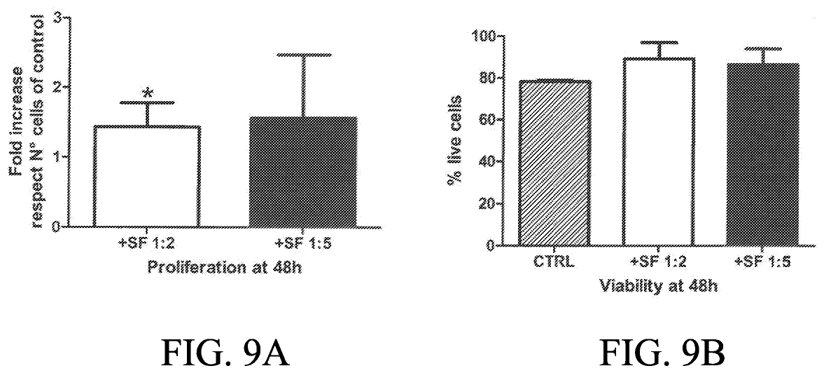

The obtained AMSC were cultured for 48 hours in the presence of different dilutions (1:2 and 1:5 with AMSC culture medium) of synovial fluid obtained from inflamed joints of patients with osteoarthritis (n=5). The diluted synovial fluid induced changes to the morphology of the AMSC, and induced an increase in proliferation rate of these cells of about 1.5 fold, without changing cell viability. FIG. 9A shows the increase in proliferation rate. FIG. 9B shows that there was no statistical change in the viability with exposure to diluted synovial fluid, relative to untreated control cells. Histograms represent means±S.-D. *P<0.05

The number and the viability of the cells was evaluated by trypan blue exclusion count.

The immunophenotype of AMSC cells exposed to synovial fluid from inflamed joints of osteoarthritis patients was also evaluated by flow cytometry. The AMSC, obtained as described above, were treated with a solution of 50% (diluted 1:1 with culture medium) of synovial fluid from inflamed joints of osteoarthritis patients (+SF1:2) (n=3).

Controls were cells that were not treated.

The treated and control AMSCs were tested for hematopoietic, and mesenchymal stem cell markers by flow cytometry analysis. The testing revealed that the treated adipose mesenchymal stem cells were negative for hematopoietic markers (CD14, CD34, CD31, CD45, HLA-DR) and positive for mesenchymal stem cells markers (CD29, CDti3, CD90, CD105, ALP). Surprisingly, the expression of staminal markers CD105, CDti3 and ALP was increased after treatment with synovial fluid. The results are presented by FIG. 10. Histograms represent means±S.D.

The data confirm that synovial fluids are able to influence adipose mesenchymal stem cells, specifically increasing the expression of stem cell markers.

In order to verify the hypothesis that a pro-inflammatory stimulus can influence chemokine production by AMSC, AMSC were cultured in presence of a 1:2 dilution of synovial fluid of patients with osteoarthritis (n=4) for 6h then the culture medium was replaced and harvested after 48h.

Chemokines production was measured by Bio-Plex® ELISA (Bio-plex Assay, Bio-Rad Laboratories) on adipose mesenchymal stem cells AMSC supernatants.

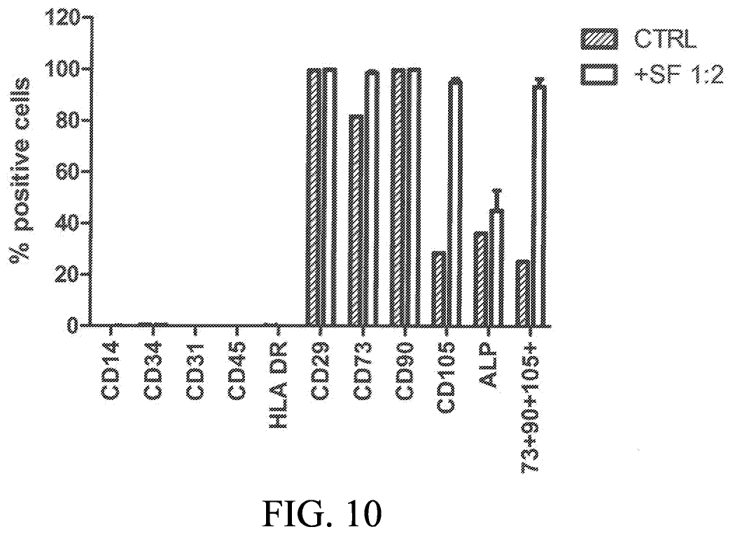

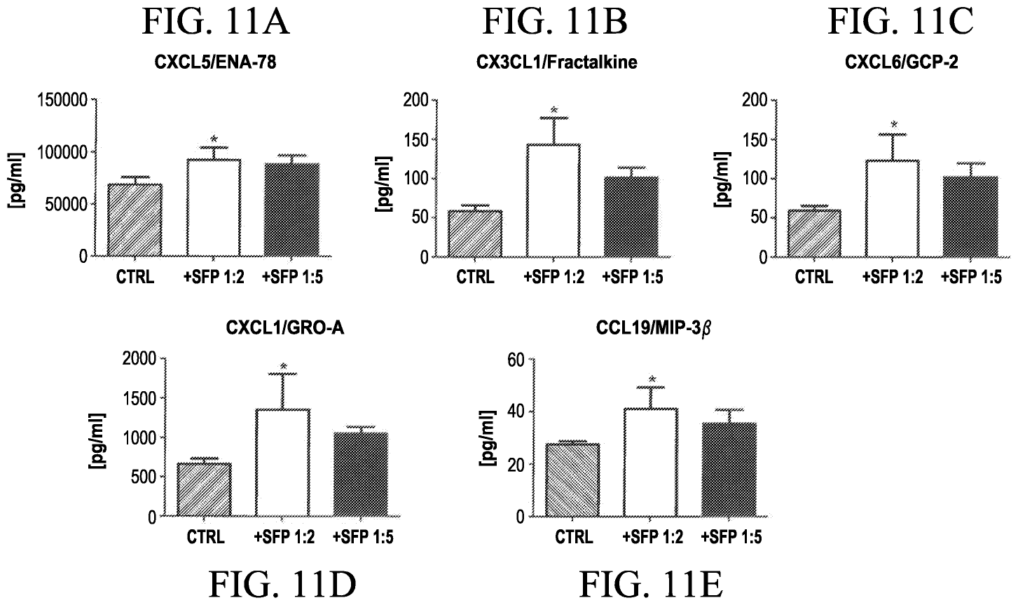

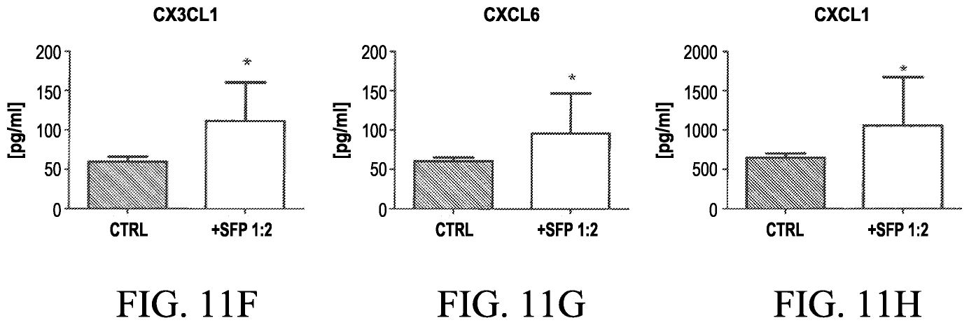

Since the synovial fluid resulted in an increase in cell proliferation, in order to better interpret the results, the chemokine production was normalized relative to cell counts at 48 hours. The results indicate that the incubation with synovial fluid stimulated the production of CXCL5, CX3CL1, CXCL6, CXCL1 and CC19 chemokines as shown by FIGS. 11A, 11B, 11C, 11D and 11E.

Data reported in FIGS. 11A through 11E were not normalized by cell count. FIGS. 11F, 11G and 11H summarize data showing cytokines that maintain a significant increase after normalization (CX3CL1, CXCL6 and CXCL1, respectively). Histograms represent means±S.D. *P<0.05. FIGS. 11F, 11G and 11E report that the cytokines produced by AMSC, cultured in the presence of a 1:2 dilution of synovial fluid, maintain a significant increase after cell count normalization.

In addition, the production of cytokines by adipose mesynchymal stem cells exposed to synovial fluid from inflamed joints of osteoarthritis patients was determined. Cytokine production was measured by Bio-Plex® ELISA (Bio-plex Assay, Bio-Rad Laboratories) on adipose mesenchymal stem cells AMSC supernatants.

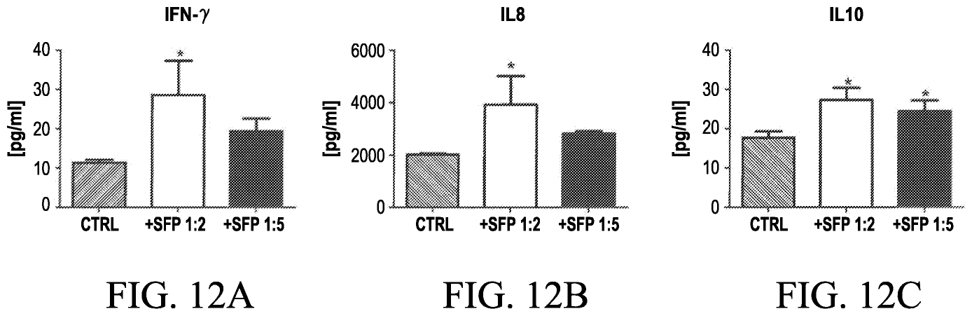

The results are presented by FIGS. 12A, 12B and 12C, for the production of INF-γ, IL-8 and IL-10, respectively. Histograms represent means±S.D. *P<0.05. As above, cytokines concentration was not normalized for cell count. After normalization no differences in cytokines production after synovial fluid treatment was found, suggesting that the increase in cytokine production is due to the increase of cell number (proliferation).

### Example 9

### Effect Of Mesenchymal Stem Cells Derived from Adipose Tissue (Amsc) on Macrophage Polarization

Monocytes were treated with GM-CSF for 7 days to induce them to differentiate to M1 macrophages (using the methods described above in Example 3).

Supernatant (conditionated medium) from AMSC were obtained by culturing cells for 48 hours in the presence of 1:2 dilution with culture medium of synovial fluid obtained from inflamed joints of patients with osteoarthritis.The M1 differentiated macrophages were cultured in the presence of the AMSC supernatant for 5 days, with conditioned medium (“CM”) of unstimulated AMSC that were treated with 50% SF for 48 h (CM+SF) or 50% synovial fluid (SF). The synovial fluid was from inflamed joints of osteoarthritis patients. Macrophages were characterized for the expression of the CD80 M1 expression marker or the CD163 M2 expression marker by flow cytometry.

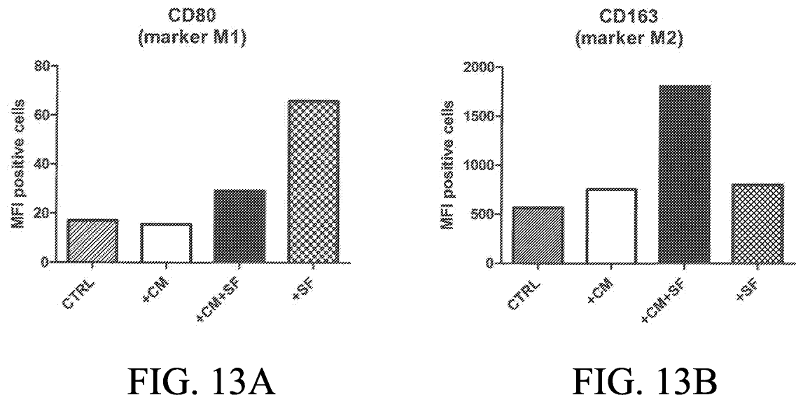

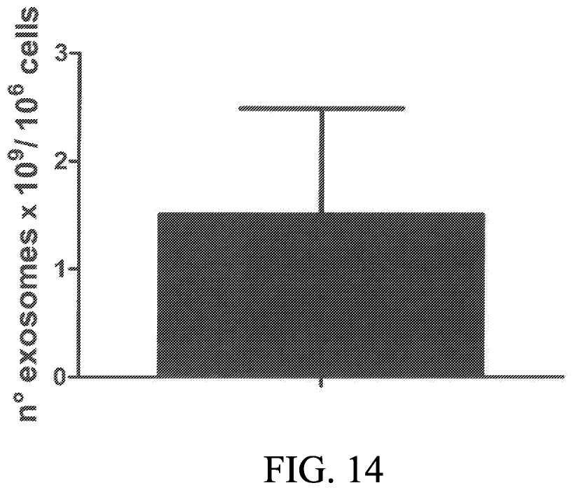

FIGS. 13A and 13B show that the synovial fluid was able to increase the differentiation toward M1 phenotype. The conditioned medium of cells treated with synovial fluid, and not that of unstimulated cells, was able to repolarize macrophages toward the anti-inflammatory M2 phenotype.

FIGS. 13A and 13B show the expression of M1 (CD80) and M2 (CD163) markers, evaluated by flow cytometry on M1 macrophages incubated in the conditions specified above. The treatment with SF increased the expression of CD80, suggesting that SF treatment is a pro-inflammatory stimulus. The treatment of M1 macrophages with conditioned medium obtained from adipose mesenchymal stem cells that were, in turn, treated with synovial fluid (CM+SF), increased the expression of the CD163 marker. This indicates that the conditionated medium of cells incubated with synovial fluid are able to repolarize macrophages. This does not happen if the conditionated medium of unstimulated stem cells (CM) is employed.

### Example 10

### Adipose Mesenchymal Stem Cells Isolation And Culture

Adipose mesenchymal stem cells (AMSCs) (n=6) were isolated from adipose tissue obtained by lipoaspirates. Lipoaspirates were enzymatically dissociated using a 0.05% collagenase II solution for 20 minutes at 37° C. (Worthington Biochemical Corporation, Lakewood, N.J., USA) and, after neutralization of the enzyme, were centrifuged at 500×g for 5 minutes and filtered through a 70 μm nylon mesh (Merck Millipore). Cells were seeded in minimum essential medium-α (MEM-α) supplemented with 10% FBS (Gibco), penicillin/streptomycin solution (10 mL/L), alanine/glutamine solution (2 mM), human epidermal growth factor (10 ng/ml), insulin solution (10 μg/ml), 2-fosfo-L-ascorbic acid, trisodium salt (100 μM) and dexamethasone (0.01 μM) (all from Sigma-Aldrich). Culture were kept at 37° C., 5% CO₂ and 95% humidity and cells were characterized by flow cytometry using MSCs positive markers (CD29, CD73, CD90 and CD105) and hematopoietic negative markers (CD34 and CD45) as described previously (R. Domenis, et al., 2015, Stem Cell Res. Ther. 6: 2. Cells were used for experiment between passage 2 and 5.

### Example 11

### Activation of Adipose Mesenchymal Stem Cells with IFNγ And TNFα

To activate AMSCs with inflammatory factors, AMSCs were seeded, as described above, at density of 15,000 cells/cm² and after 24 hours supernatant was replaced with fresh culture medium supplemented with 5% certified exosomes-free serum (Gibco) with recombinant human IFNγ and TNFα (Prepotech) at different concentrations (10, 20 and 40 ng/ml). The concentration 10 ng/ml corresponds to 200 U/ml. After 48 hours, AMSCs were harvested and cell proliferation/viability was determined by trypan blue exclusion assay. For flow cytometry analysis, AMSCs were fixed and permeabilized with intracellular Fix/Perm solution (eBiosciences), incubated with FITC-conjugated indoleamine-pyrrole 2,3-dioxygenase (IDO) antibody (eBiosciences) for 15 min and then washed twice with PBS. Flow cytometry was carried out on the FACSCalibur (Becton Dickson) and data analysed using Flowing software. Supernatants were also harvested, centrifuged for 10 minutes at 14,000×g and stored at −80° C. for exosomes isolation or cytokines detection. The concentration of IL-6, IL-10, IL-8 and CCL-2 was determined with a magnetic beads-based multiplex assay (Bio-plex Assay, Bio-Rad Laboratories), while prostaglandin E₂ release (PGE₂) was quantified with an enzyme-linked immunosorbent assay (ELISA) kit (Invitrogen).

### Example 12

### Amscs-Derived Exosomes Isolation and Characterization

Exosomes were isolated from AMSCs supernatants by polymer precipitation methods with (ExoQuick-TC System Biosciences) as described previously (R. Domenis, et al., 2017, PLoS One. 12, e0169932). The exosomes-containing pellet was resuspended in PBS buffer or lysis buffer for subsequent analysis. AMSCs-derived exosomes number was determined using the Exocet kit (System Biosciences), according to manufacturer's instructions. Briefly, exosomes were lysed using a gentle lysis solution to preserve the enzymatic activity of the exosomal Acetylcholinesterase (AChE) enzyme. A standard curve was generated using known numbers of exosomes (as measured by NanoSight) and calibrated with a recombinant AChE enzyme standard solution provided in the kit.

The size distribution of the collected exosomes was determined using a qNano (Izon) nanopore-based exosome detection system, according to the manufacturer's instructions.

AMSCs lysate, AMSCs-derived exosomes and Exoquick-derived supernatants were analysed for the expression of exosomal markers and contaminants by immunoblotting. Anti-CD9 (1:1000, System BioScience), anti-CD63 (1:1000, LS Bio), anti-CD81 (1:500, Abcam), anti-TSG101 (1:500, Abcam), anti-calnexin (1:1000 Enzo Life Technologies), anti-GRP94 (1:1000 Genetex) and anti-RISC (1:1000 Abcam) were used as primary antibodies. Horseradish peroxidase (HRP)-conjugated IgG antibody (1:1000, Dako) was used as the secondary antibody. To ensure that IFNγ and TNFα were not present in our exosome preparation as contaminants, the culture medium supplemented with cytokines at different concentrations (10, 20 and 40 ng/ml) was incubated with Exoquick as described above and concentration of IFNγ and TNFα was measured by magnetic beads-based multiplex assay.

### Example 13

### Exosomal RNA Isolation and Rt-Pcr

To preserve small RNAs, total RNA was extracted from 5×10⁹ AMSCs-derived exosomes, using mirVana PARIS Kit (Life Technologies), adding cel-miR39 spike-in as exogenous control (ThermoFisher Scientific). Extracted RNA quality and quantity was evaluated by NanoDrop™ 1000 Spectrophotometer (ThermoFisher Scientific) and was stored at −80° C. until use.

The relative concentrations of miRNAs involved in regulation of macrophages M1 (has-miR-21-5p, has-miR-127-3p and has-miR-155-5p) and M2 (has-miR-34a-5p, has-miR124-3p, has-miR135b-5p and hsa-miR146a-5p) polarization were assessed using TaqMan® Advanced miRNA Assays (ThermoFisher Scientific), according to manufacturer's instructions, except for cDNA templates that were diluted 1:2 instead of recommended 1:10.

Real-time reaction was performed on the Applied Biosystems QuantStudio 3 System. MiRNA relative concentrations were normalized using relative standard curve method obtained by serial dilutions of cel-miR39 (1 nM-100 fM).

### Example 14

### Monocytes Isolation and Differentiation into M1 Macrophages

Human peripheral blood mononuclear cells (PBMCs) were isolated from EDTA-uncoagulated blood of blood donors by Ficoll gradient centrifugation (Millipore). Monocytes were separated from PBMCs by negative selection using a human CD14+ cell enrichment kit (StemCell Technologies) according to the manufacturer's instructions and resuspended in RPMI medium supplemented with 10% heat inactivated fetal bovine serum (FBS), 1% glutamine, 1% pyruvate, 1% non-essential aminoacid, 1% penicillin/streptomycin, 1% Hepes (all from Euroclone). To remove the exosomal fraction present in FBS, serum was ultracentrifuged for 4 hours at 100,000×g. Purity of monocytes was over 95% as judged by staining with anti-CD14 (eBiosciences) (data not shown).

For macrophages differentiation, CD14+monocytes were seeded in multiwell plates at 5×10⁵/cm² in complete RPMI medium supplemented with 100 ng/ml granulocyte macrophage-colony stimulating factor (GM-CSF, Peprotech) in presence of 8×10⁸ AMSC-derived exosomes; medium was changed completely every 3 days. On day 9, macrophages was harvested with TrypLE™ express detachment solution (Gibco) and characterized by flow cytometry for the expression of M1 and M2 macrophages markers using CD80, CD206 and CD163 antibodies (eBiosciences). Macrophages were also lysed in RIPA buffer and expression of IRAK1, Notchl and SIRPb1 was analysed by immunoblotting. Anti-IRAK1 (1:1000, Cell Signalling), anti-β-actin (1:5000, Cell Signalling), anti-Notchl (1:500, Cell signalling) and anti-Sirp-β1 (1:200, Santa Cruz Biotecnologies) were used as primary antibodies. Horseradish peroxidase (HRP)-conjugated IgG antibody (1:1000, Dako) was used as the secondary antibody.

### Example 15

### Statistical Methods and Results

Data are reported as mean±standard deviation. Statistical analysis has been performed using GraphPad Software (version 4.0c). Data were tested for normal distribution using the Kolmogorov-Smirnov test. Repeated measurements were analysed by one-way analysis of variance followed by the Bonferroni or Dunnett post test. P values <0.05 was considered significant.
A. Effect of the Stimulation with Cytokines IFNγ and TNFα on AMSCs.

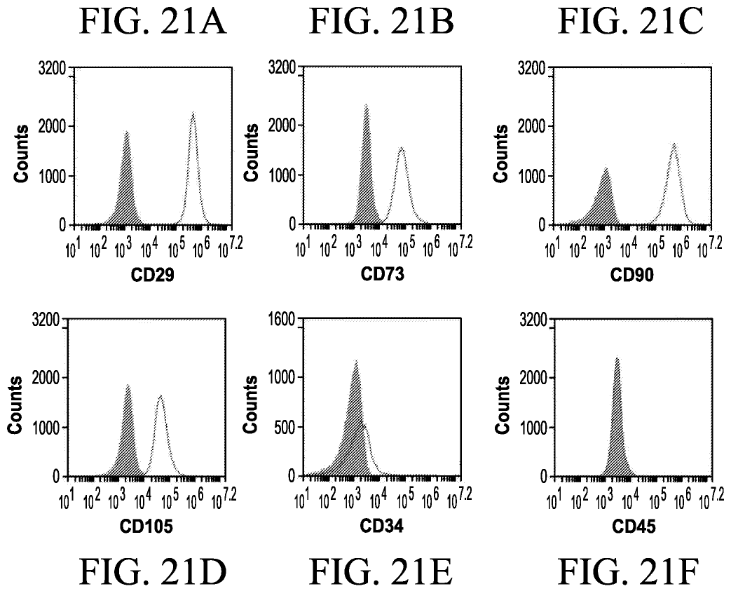

AMSCs isolated from adipose tissues were tested using specific surface markers by flow cytometry: the tested AMSCs were almost completely negative for the hematopoietic markers (CD34 and CD45) and >95% positive for the mesenchymal stem cells markers (CD29, CD73, CD90 and CD105) (FIG. 21A-21F).

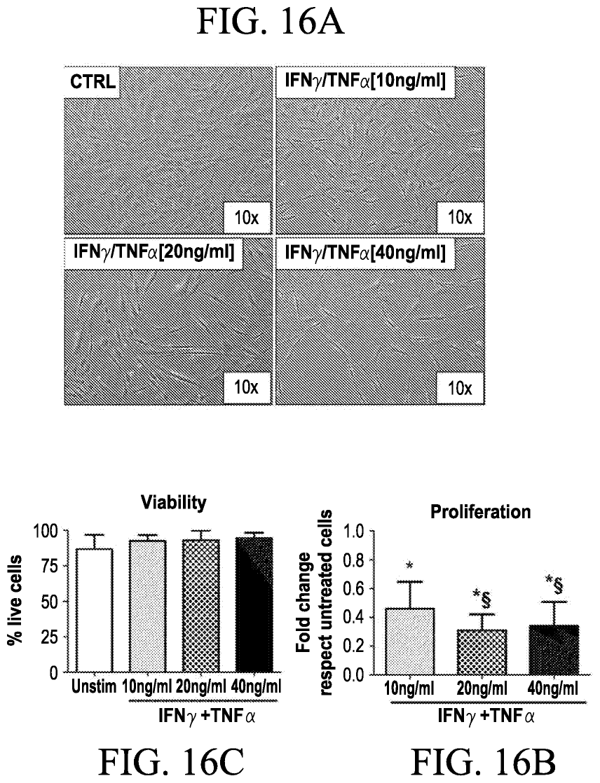

To determine whether inflammatory stimuli may affect morphology and viability of AMSCs, cells were cultured in the presence of IFNγ/TNFα at concentration of 10, 20 and 40 ng/ml for 48 hours. We observed that the treatment with cytokines induced morphological changes, since the cells become more elongated and are characterized by an irregular shape (FIG. 16A). The incubation with cytokines decreased AMSCs proliferation in a concentration-dependent manner (FIG. 16B), while cells viability was not affected (FIG. 16C).

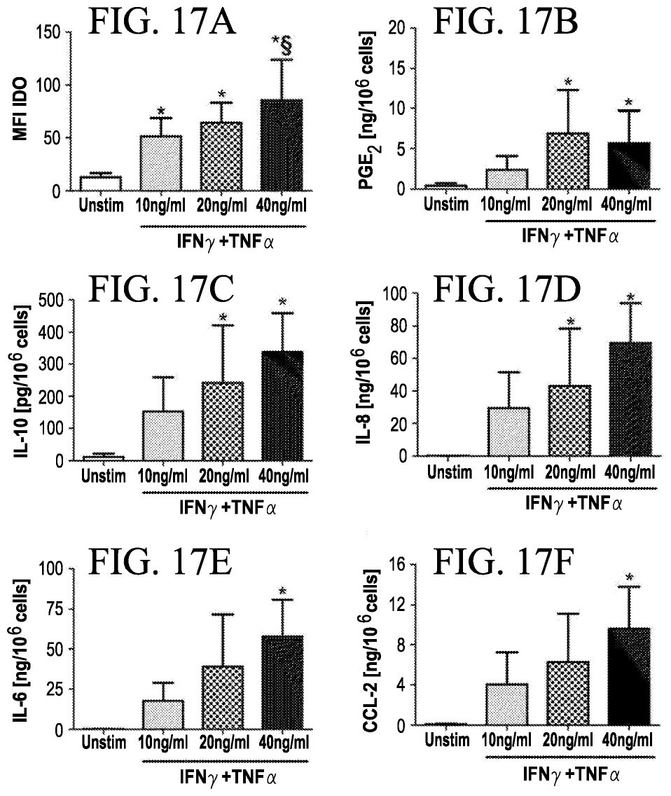

We next examined the effect of inflammatory stimuli on the release of immunosuppressive factors and cytokines/chemokines by AMSCs. Treatment with IFNγ/TNFα increased the expression of the enzyme indoleamine-pyrrole 2,3-dioxygenase (IDO) in a concentration-dependent manner (FIG. 17A), while the release of PGE₂ (FIG. 17B), IL-10 (FIG. 17C) and IL-8 (FIG. 17D) was significantly induced only after treatment with at least 20 ng/ml of the IFNγ/TNFα mixture. Finally, IL-6 (FIG. 17E) and CCL-2 (FIG. 17F) upregulation was significant only after treatment with 40 ng/ml of IFNγ/TNFα.
B. Characterization of AMSCs Derived-Exosomes After Stimulation by Pro-Inflammatory Cytokines

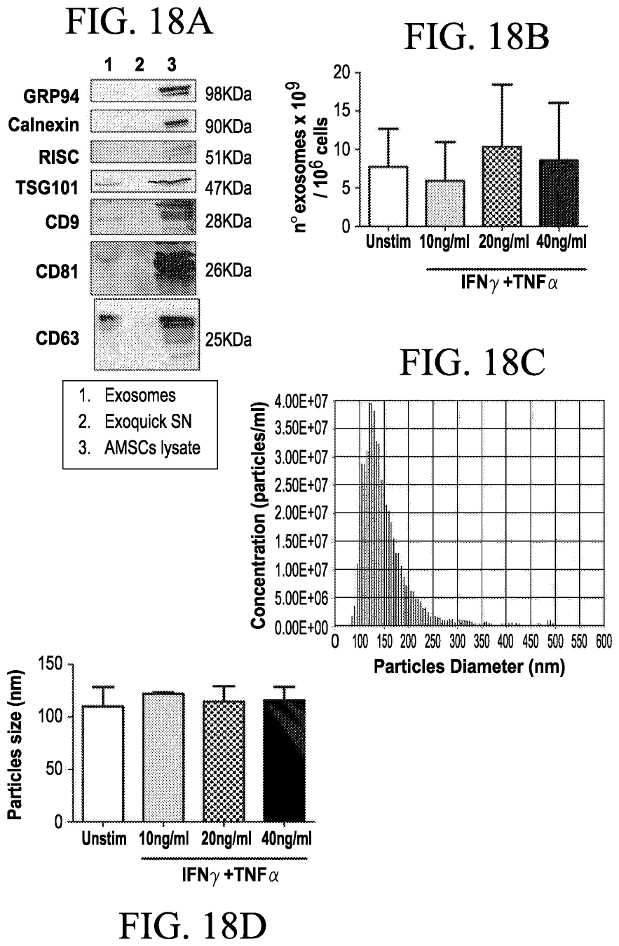

AMSCs were pre-treated with IFNγ/TNFα at increasing concentration (10, 20 and 40 ng/ml), then an enriched fraction of exosomes was obtained from the supernatants using the Exoquick polymer-based strategy. As shown in FIG. 18A, AMSCs-derived exosomes and AMSCs lysates expressed the specific exosomal markers CD9, CD63, CD81 and TSG101, while no signal was observed for Exoquick-derived supernatant samples, that were used as negative control. In order to evaluate the impurities in our exosome preparations, we also evaluated the expression of proteins associated with subcellular compartments, which are supposed to be absent or under-represented in exosomes. Our data showed lack of calnexin (endoplasmic reticulum protein) and RISC complex (nucleus protein) in exosome fraction, indicating successful enrichment. We also reported a faint band for GRP94 (endoplasmic reticulum protein) in the exosomal fraction probably due to a slight contamination by apoptotic bodies.

The concentration of AMSCs-derived exosomes was determined measuring the activity of AChE by Exocet kit. As reported in FIG. 18B, the mean concentration of exosomes released by untreated cells was 7.6±2.6×10⁹ per million of producing cells. Treatment with cytokines did not influence the number of exosomes released by AMSCs.

Finally, the average size of the collected vesicles, determined by qNano technology, as described above, was 115±11.5 nm, in range with exosomes proper size, and was not influenced by AMSCs cytokines treatment (FIG. 18D).
C. Exosomes Derived from AMSCs Pre-Activated with Pro-Inflammatory Cytokines Induce an Anti-Inflammatory M2 Phenotype Reverting M1 Differentiation.

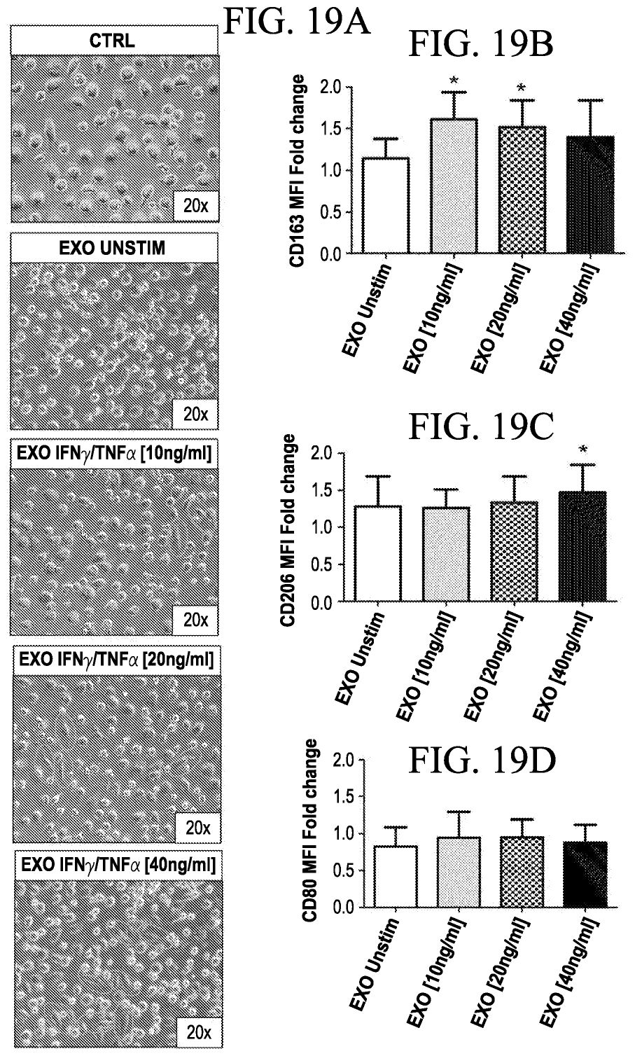

To examine the ability of AMSCs-derived exosomes in inducing anti-inflammatory phenotype in macrophages, CD14+ monocytes isolated from PBMCs of blood donors were induced to differentiate into M1 macrophages with GM-CSF in presence of exosomes isolated from supernatants of AMSCs pre-activated with IFNγ/TNFα. As shown in FIG. 19A, at day 9, control monocytes gave rise to “fried egg-shaped” morphology, a typical feature of M1-like macrophages. When monocytes were differentiated in the presence of exosomes obtained from pre-activated AMSCs, some cells displayed an elongated, spindle-like morphology, a typical feature of M2 macrophages (F.Y. McWhorter, et al., 2013, Proc. Natl. Acad. Sci. 110: 17253-17258). The effect is particularly evident in monocytes incubated with exosomes isolated from AMSCs pre-activated with 40 ng/ml of IFNγ/TNFα. Indeed, compared to untreated M1-like macrophages, only exosomes isolated from pre-activated AMSCs are able to upregulate the expression of the M2 macrophage marker CD163 (FIG. 19B). With regard to CD206 expression, it became significant only after treatment with exosomes produced by pre-activated cells with 40 ng/ml of IFNγ/TNFα (FIG. 19C). In contrast, the expression of the M1 macrophage marker CD80 did not change significantly in the presence of AMSCs-derived exosomes (FIG. 19D).

In order to evaluate a possible contamination of IFNγ and TNFα in exosome preparations, the culture medium supplemented with cytokines at different concentrations (10, 20 and 40 ng/ml) was treated with Exoquick and concentration of IFNγ and TNFα was measured by magnetic beads-based multiplex assay.

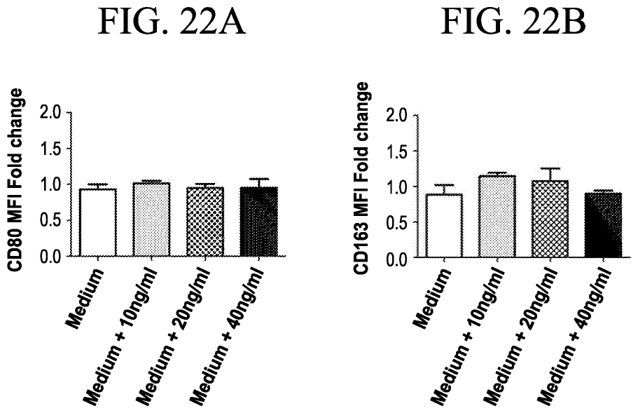

We found slightest amounts of IFNγ (0.096±0.003 pg/ml) and TNFα (0.039±0.001 pg/ml), which are minimal compared to those used in literature to stimulate monocytes or macrophages. Moreover, we evaluated the potential effect of these contaminants during monocytes differentiation, finding that they did not influence macrophages polarization in the expression of CD80 (FIG. 22A) and CD163 (FIG. 22B).
D. Exosomes Derived from AMSCs Pre-Activated with Inflammatory Cytokines Contained miRNA Involved in M2 Macrophages Polarization

To analyse the expression of miRNAs involved in the regulation of macrophage polarization, total RNA was extracted from AMSCs-derived exosomes (K. Essandoh, et al., 2016, SHOCK. 46: 122-131. We evaluated the expression of miRNAs regulating the differentiation towards M1 (miR-127-3p and miR-155-5p) or M2 (miR-34a-5p, miR124-3p, miR135b-5p and miR146a-5p) phenotypes. Of note, miR-21-5p is able to redirect both M1 and M2 polarization, depending on protein target. In unstimulated AMSCs-derived exosomes, all the miRNAs under investigation, except for 124-3p (data not shown), were expressed at low level.

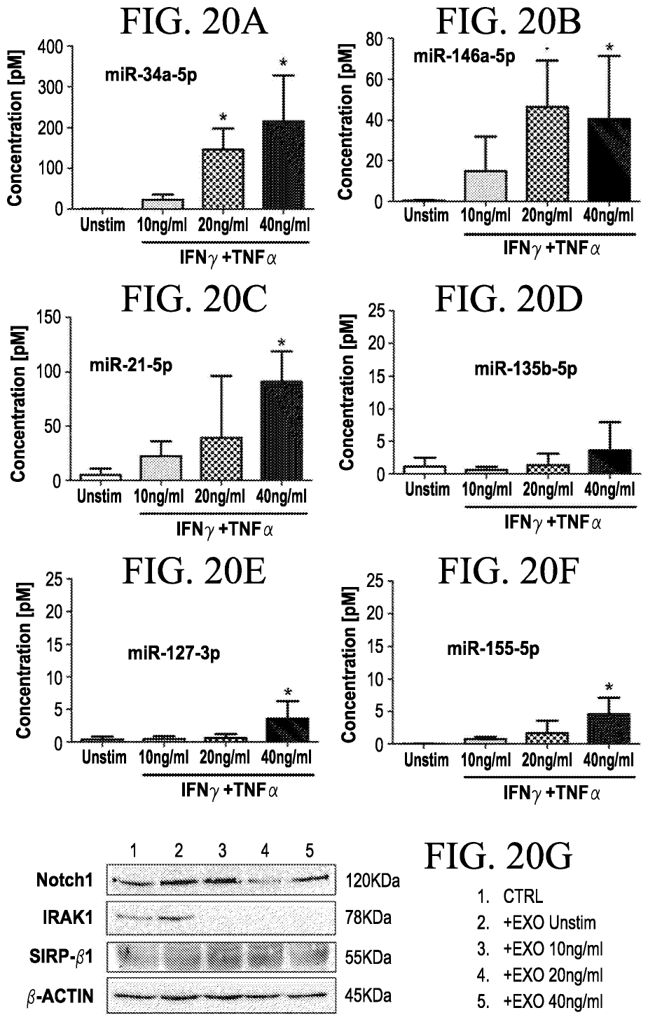

The expression of miRNA-34 (FIG. 20A) and miRNA-146 (FIG. 20B) were significantly higher in exosomes produced by AMSCs pre-activated with 20 and 40 ng/ml IFNγ/TNFα compared to those of untreated cells, while miRNA-21 expression was significantly upregulated only for 40 ng/ml cytokine pre-stimulation (FIG. 20C). No difference was observed for the expression of miR-135 (FIG. 20D). The expression of miR-127 (FIG. 20E) and miR-155 (FIG. 20F) were significantly increased only in exosomes produced by AMSCs activated with the highest (40 ng/ml) cytokine concentration, but at a very lesser extent compared to the other described miRNAs.

Finally, in order to evaluate the downstream effect of miRNA expression upregulation in our AMSC experimental model, we choose to analyse the protein expression of some specific miRNA targets in macrophage lysates. In particular, we analysed the expression of Notch1, IRAK1 and Sirp-β1 targeted by miR-34a (P. Jiang, et al., 2012, Exp. Cell Res. 318: 1175-1184), miR-146 (K. D. Taganov, et al., 2006, Proc. Natl. Acad. Sci. USA, 103: 12481-6) and miR-21 (C. I. Caescu, et al., 2015, Blood 125: e1-13), respectively.

As illustrated in FIG. 20G, IRAK1 expression was dramatically reduced after treatment with exosomes of pre-stimulated AMSCs, while the expression of Notch1 was reduced only with exosomes release from cells treated with 20 ng/ml of cytokines. The expression of Sirp-β1 was not affected by the treatment with exosomes.

### INCORPORATION BY REFERENCE

All publications cited herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

We claim:

1. A method of producing anti-inflammatory exosomes capable of inhibiting inflammation in a subject diagnosed with an inflammatory disease or disorder, the exosomes produced by a process comprising:
a) culturing adult stem cells in a suitable culture medium, for a time period sufficient for the adult stem cell culture medium to accumulate a useful concentration of anti-inflammatory exosomes, wherein the culture medium comprises an activating composition,
(b) collecting the culture medium of (a), and
(c) isolating anti-inflammatory adult stem cell exosomes from the culture medium collected in (b);
wherein the activating composition comprises at least one of:
(i) fluid extracted from inflamed tissue or other anatomical structure of a mammal diagnosed with the inflammatory disease or disorder,
(ii) blood, plasma or serum from a mammal diagnosed with the inflammatory disease or disorder,
(iii) the fluid of (i) or (ii) further comprising at least one cytokine at a concentration sufficient to enhance the effectiveness of the activating composition comprising (i) or (ii),
(iv) one or more cytokines at a concentration sufficient to induce the adult stem cells to secrete anti-inflammatory exosomes capable of inhibiting inflammation in a mammal, and the activating composition excludes the fluid of (i) and/or the blood, plasma or serum of (ii).

2. The method of claim 1, wherein the at least one cytokine of (iii) or (iv) is selected from the group consisting of interferon-gamma (IFNγ), interleukin-1α (IL-1α), interleukin-1-β (IL-1β), interleukin-6 (IL-6), interleukin 12b (IL-12b), tumor necrosis factor-α (TNFα) and combinations thereof.

3. The method of claim 2, wherein the cytokine of (iii) or (iv) is IFNγ and/or TNFα.

4. The method of claim 1, wherein the at least one cytokine of (iii) or (iv) is present in a concentration ranging from about 10 ng/ml to about 50 ng/ml.

5. The method of claim 3, wherein the at least one cytokine is present in a concentration ranging from about 10 ng/ml to about 40 ng/ml.

6. The method of claim 1, wherein the time period for the culturing of step (a) ranges from about 1 to about 6 days.

7. The method of claim 1, wherein the time period for the culturing of step (a) ranges from about 3 to about 6 days.

8. The method of claim 1, wherein the step of isolating the anti-inflammatory adult stem cell exosomes from the culture medium of (a) is selected from the group consisting of polymer precipitation, immunological separation, magnetic immunocapture and combinations thereof.

9. The method of claim 1, wherein the step of isolating the anti-inflammatory adult stem cell exosomes from the culture medium of (a) is selected from the group consisting of ultracentrifugation, density gradient centrifugation, size exclusion chromatography, ultrafiltration and combinations thereof.

10. The method of claim 1, wherein the subject is a mammal is selected from the group consisting of a human, a canine, a feline, a porcine, and an equine.

11. Isolated and purified anti-inflammatory exosomes produced by the method of claim 1.

12. The isolated and purified anti-inflammatory exosomes of claim 11, comprising at least one miRNA selected from the group consisting of miRNA-34, miRNA-146 and miR-127.

13. The isolated and purified anti-inflammatory exosomes of claim 11, wherein the exosomes comprise miRNA and the miRNA content of the exosomes consists essentially of at least one miRNA selected from the group consisting of miRNA-34, miRNA-146 and miR-127.

14. A method of treating a subject diagnosed with an inflammatory disease or disorder, comprising administering to the subject an effective amount of the anti-inflammatory exosomes of claim 11.

15. The method of claim 14, wherein the inflammatory disease or disorder is a tissue specific disease or disorder.

16. The method of claim 14, wherein the inflammatory disease or disorder is selected from the group consisting of the inflammatory diseases resulting from metabolic syndrome, inflammatory diseases of the gastrointestinal system, inflammatory diseases of the pulmonary system, inflammatory diseases of the skin, inflammatory diseases of the musculature, inflammatory diseases of the joints, and inflammatory diseases of the nervous system.

17. The method of claim 14, wherein the inflammatory disease or disorder is selected from the group consisting of coronary artery disease, chronic obstructive pulmonary disease (COPD), asthma, bronchitis, inflammatory bowel disease (IBD), Alzheimer's disease, Parkinson's disease, polymyositis, dermatomyositis, inclusion body myositis (IBM), juvenile myositis, rheumatoid arthritis, osteoarthritis, amyloidosis, ankylosing spondylitis, bursitis, psoriatic arthritis, and Still's disease.

18. The method of claim 14, wherein the anti-inflammatory exosomes are administered by a route selected from the group consisting of, intravenous injection, intramuscular injection, intraarticular injection or infusion, subcutaneous inection, and intrathecal injection or infusion.

19. The method of claim 14, wherein the inflammatory disease or disorder is osteoarthritis, and the method comprises injecting an effective amount of the anti-inflammatory exosomes into one or more inflamed joints of the mammal.

20. The method of claim 14, wherein the inflammatory disease or disorder is pulmonary, and the method comprises administering the anti-inflammatory exosomes as an inhaled mist or aerosol.

21. The method of claim 14, wherein the subject is a mammal selected from the group consisting of a human, a canine, a porcine, a feline and an equine.

22. The method of claim 14, wherein the anti-inflammatory exosomes are administered systemically, in an amount ranging from about 1.5×10¹⁰ to about 1.5×10¹³ exosome particles per kilogram of total body weight.

23. The method of claim 14, wherein the anti-inflammatory exosomes are administered in an amount ranging from about 1.5×10¹⁰ and 1.5×10¹¹ exosome particles injected or infused into a localized tissue or anatomical space.

24. The method of claim 14, further comprising co-treating the mammal with at least one additional anti-inflammatory agent, the at least one additional anti-inflammatory agent selected from the group consisting of a steroidal anti-inflammatory, a non-steroidal anti-inflammatory, an anti-inflammatory anti-TNF alpha antibody and combinations thereof.

25. A pharmaceutical composition comprising the exosomes of claim 11, and a physiologically acceptable carrier.
